(19) United States
(12) Patent Application Publication
(10) Pub. No.: US 2025/0283882 A1
(43) Pub. Date: Sep. 11, 2025

(54) HIGH SENSITIVITY DNA LINKED IMMUNOSORBENT SIGNAL AMPLIFICATION ASSAY (DLISA) FOR DETECTION OF INFECTIOUS SARS-COV-2 VIRUS AND VARIANTS

(71) Applicant: ARIZONA BOARD OF REGENTS ON BEHALF OF ARIZONA STATE UNIVERSITY, Scottsdale, AZ (US)

(72) Inventors: Nicholas Stephanopoulos, Scottsdale, AZ (US); Yang Xu, Chandler, AZ (US); Shaopeng Wang, Chandler, AZ (US); Hao Yan, Gilbert, AZ (US)

(21) Appl. No.: 18/701,890

(22) PCT Filed: Oct. 20, 2022
(86) PCT No.: PCT/US2022/078432

§ 371 Date: Apr. 16, 2024

Related U.S. Application Data
(60) Provisional application No. 63/257,838, filed on Oct. 20, 2021.

(51) Int. Cl. G01N 33/569; G01N 33/53

(52) U.S. Cl. CPC G01N 33/56983; G01N 33/5308; G01N 2333/165; G01N 2458/10; G01N 2469/10

(57) ABSTRACT

The present disclosure relates to the use of DNA-peptide hybrid molecules to detect target molecules in a sample. In some embodiments, the DNA-peptide hybrid molecules comprise target-specific binding peptides which selectively bind to a target molecule. Kits comprising DNA-peptide hybrid molecules are also provided.

### CROSS-REFERENCE TO RELATED APPLICATIONS

The present application is the U.S. National Stage of PCT/US2022/078432 with international filing date of Oct. 20, 2022 and which published as WO 2023/070034 on Apr. 27, 2023, and which claims priority to U.S. Provisional Patent Application No. 63/257,838, filed Oct. 20, 2021, the entire contents of each of which are hereby incorporated by reference.

### STATEMENT REGARDING FEDERALLY SPONSORED RESEARCH

This invention was made with government support under GM132931 awarded by the National Institutes of Health. The government has certain rights in the invention.

### REFERENCE TO AN ELECTRONIC SEQUENCE LISTING

The contents of the electronic sequence listing (112624.01358.xml; Size: 7,030 bytes; and Date of Creation: Apr. 18, 2024) is herein incorporated by reference in its entirety.

### FIELD OF INVENTION

The present disclosure relates generally to methods and compositions for the detection of molecules in a subject sample. In some embodiments, the methods and compositions include a DNA-peptide hybrid molecule, wherein the peptide specifically binds to a target of interest, such as peptide antigen or epitope of a pathogen. In some embodiments, the molecule comprises a portion of the pathogen SARS-CoV-2.

### BACKGROUND

Severe acute respiratory syndrome coronavirus 2 (SARS-CoV-2) has rapidly spread across the globe and infected more than 200 million individuals (covid19.who.int). Nucleic acid tests and antibody responses tests are widely applied to diagnose coronavirus disease 2019 (Covid-19). The nucleic acid tests primarily detect the SARS-CoV-2 RNA genome. The SARS-CoV-2 virus expresses a large (140 kDa) glycoprotein termed spike protein (S, A homotrimer), which involves binding to host cells via the receptor angiotensin-converting enzyme 2 (ACE2). The spike protein with the receptor-binding domain (RBD), the target of ACE2 and many neutralizing antibodies, is highly immunogenic. However, since SARS-CoV-2 viruses mutate with high frequency, it is common to find multiple variants of the COVID-19 pandemic spreading widely, such as Alpha (B1.1.7) from the UK, Beta (B1.351) from South Africa, Gamma (P1) from Brazil, and Delta (B1.617.2) from India, and so on. These lineages are each characterized by numerous mutations in the spike protein, raising concerns that they are not affected by neutralizing monoclonal and vaccine-induced antibodies. Emerging SARS-CoV-2 variants can be problematic as they can result in changes that make the virus more transmissible, pathogenic, more likely to escape to treatment using neutralizing antibodies, more resistant to vaccines, and able to evade diagnostic tests.

Diagnostic tests for SARS-CoV-2 infection belong to three categories: (1) nucleic acid amplification tests, which detect the presence of virus RNA by reverse transcription-polymerase chain reaction (RT-PCR); (2) tests detecting the presence of viral antigens; and (3) tests detecting the presence of serological antibodies against SARS-Cov-2 antigens. The World Health Organization (WHO) recommends nucleic acid detection of SARS-CoV-2 in respiratory samples for the diagnosis of the virus. However, there remains a great need for detection assays that measure viral antigens and variants.

Detection assays for SARS-CoV-2 are becoming available, including ELISA, lateral flow assays, and virus neutralization assays. SARS-CoV-2 antigen-detecting rapid diagnostic tests (Ag-RDTs) provide potent tools for pathogen detection at the point of care and facilitate public health intervention. Nonetheless, the majority of Ag-RDT validation studies were done before the emergence and subsequent dominance of SARS-CoV-2 variants of concern (VOC). For these variants, there is evidence of an increase in transmissibility, more severe disease (e.g., increased hospitalizations or deaths), a significant reduction in neutralization by antibodies generated during previous infection or vaccination, reduced effectiveness of treatments or vaccines, and failure of diagnostic detection (www.cdc.gov/coronavirus/2019-ncov/variants/). Currently, there are few regular diagnostics for SARS-CoV-2 variants of concern (VOCs). A recent study compared seven commercially available SARS-CoV-2 antigen diagnostic tests against an established RT-PCR assay. The reports showed that the sensitivity range of most antigen tests overlaps with SARS-CoV-2 viral loads observed in the early week of symptoms in the infectious period in most patients. All of the Ag-RDT is based on the antibody-dependent assay.

### SUMMARY

In an aspect of the current disclosure, methods of detecting the presence of SARS-CoV-2 in a sample from a subject are provided. In some embodiments of the methods, the methods comprise: i) contacting the sample to a capture molecule, the capture molecule comprising a nanobody specific for SARS-CoV-2, wherein the capture molecule is linked to a solid support; ii) incubating the sample in the presence of the capture molecule under conditions for SARS-CoV-2 in the sample to bind to the capture molecule, thereby forming a “V-AB” complex; iii) contacting the V-AB complex with a detection molecule under conditions to allow the detection molecule to bind the V-AB complex, the detection molecule comprising a DNA-peptide hybrid molecule, the DNA-peptide hybrid molecule comprising a DNA nanostructure chemically linked to one or more target-specific binding peptides, wherein the target-specific binding peptides specifically binds SARS-CoV-2; iv) detecting the presence of SARS-CoV-2 in the sample based on the presence of the bound detection molecule. In some embodiments of the methods, the DNA nanostructure of the detection molecule comprises one of: a single-stranded DNA molecule, a three-helix bundle, a four-helix bundle, a six-helix bundle, a triangular DNA origami structure, a tetrahedral wireframe cage, a block-like origami cuboid, reconfigurable tweezers, double crossover tiles, branched three-way junctions, and a three-legged stool. In some embodiments of the methods, the DNA nanostructure is linked to more than one target-specific binding peptide. In some embodiments of the methods, the DNA nanostructure is linked to three target-specific binding peptides. In some embodiments of the methods, one or more of the target-specific binding peptides comprises LCB1, target specific binding peptides with the sequence DKEWILQKIYEIMRLLDELGHAEASMRVSDLIYEFMKKGDERLLEEAERLLEEVER (SEQ ID NO: 2). In some embodiments of the methods, the SAB comprises a nanobody that specifically binds to the N-terminal domain of the SARS-CoV-2 spike protein. In some embodiments of the methods, the detection molecule comprises a fluorescent molecule. In some embodiments of the methods, the detecting of step iv) comprises detecting a fluorescent signal from the detection molecule, wherein the presence of the fluorescent signal from the detection molecule denotes the presence of SARS-CoV-2 in the sample. In some embodiments of the methods, the detecting of step iv) comprises detecting the presence of the detection molecule using an automated reader or a smartphone. In some embodiments of the methods, the solid support comprises a microplate. In some embodiments of the methods, the solid support comprises a microfluidic device. In some embodiments of the methods, the solid support comprises a bead. In some embodiments of the methods, the DNA nanostructure comprises a single stranded DNA molecule. In some embodiments of the methods, the single-stranded DNA molecule comprise fluorescent labels. In some embodiments of the methods, detection comprises a primer exchange reaction (PER). In some embodiments of the methods, the detection further comprises contacting the detection molecule with fluorescently labeled oligonucleotides that hybridize with the product of the PER. In some embodiments of the methods, the target-specific binding peptide binds SARS-CoV-2 alpha, beta, gamma, and delta spike protein variants. In some embodiments of the methods, the method further comprises treating the subject based on the detection of SARS-CoV-2 in the sample.

In another aspect of the present disclosure, kits for detecting the presence of SARS-CoV-2 in a sample are provided. In some embodiments of the kits, the kits comprise: i) a capture molecule linked to a solid support, wherein the capture molecule is a nanobody specific for SARS-CoV-2; ii) a detection molecule comprising a DNA nanostructure linked to one or more target-specific binding peptides, wherein the one or more target-specific binding peptides bind SARS-CoV-2. In some embodiments of the kits, the kits further comprise a detection reagent. In some embodiments of the kits, the DNA nanostructure is linked to more than one target-specific binding peptides. In some embodiments of the kits, the DNA nanostructure is linked to three target-specific binding peptides. In some embodiments of the kits, one or more of the target-specific binding peptides are LCB1. In some embodiments of the kits, the DNA nanostructure comprises a fluorescent molecule. In some embodiments of the kits, the capture molecule binds to the N-terminal domain of the SARS-CoV-2 spike protein. In some embodiments of the kits, the solid support comprises a microplate. In some embodiments of the kits, the solid support comprises microfluidic device. In some embodiments of the kits, the solid support comprises a bead. In some embodiments of the kits, the DNA nanostructure comprises a single-stranded DNA molecule. In some embodiments of the kits, the kit comprises components for a primer extension reaction, comprising DNA hairpin probes, and optionally, a polymerase, and labeled nucleotides. In some embodiments of the kits, the target-specific binding peptide binds SARS-CoV-2 alpha, beta, gamma, and delta spike protein variants.

In a further aspect of the present disclosure, methods of detecting the presence of SARS-CoV-2 in a sample from a subject are provided. In some embodiments of the methods, the method comprises: i) contacting the sample to a capture molecule, the capture molecule comprising a DNA-peptide hybrid molecule, the DNA-peptide hybrid molecule comprising a DNA nanostructure chemically linked to one or more target-specific binding peptides, wherein the capture molecule is linked to a solid support; ii) incubating the sample in the presence of the capture molecule under conditions for SARS-CoV-2 in the sample to bind to the capture molecule, thereby forming a “V-AB” complex; iii) contacting the V-AB complex with a detection molecule under conditions to allow the detection molecule to bind the C-AB complex, the detection molecule comprising a SARS-CoV-2 specific binding molecule (SBM); iv) detecting the presence of SARS-CoV-2 in the sample based on the presence of the bound detection molecule. In some embodiments of the methods, the detection molecule comprises a nanobody specific for SARS-CoV-2. In some embodiments of the methods, the DNA nanostructure is selected from the group consisting of: a single-stranded DNA molecule, a three-helix bundle, a four-helix bundle, a six-helix bundle, a triangular DNA origami structure, a tetrahedral wireframe cage, a block-like origami cuboid, reconfigurable tweezers, double crossover tiles, branched three-way junctions, and a three-legged stool. In some embodiments of the methods, the DNA nanostructure is linked to more than one target-specific binding peptide. In some embodiments of the methods, the DNA nanostructure is linked to three target-specific binding peptides. In some embodiments of the methods, one or more of the target specific binding peptides are LCB1. In some embodiments of the methods, the capture molecule binds to the receptor binding domain of the SARS-CoV-2 spike protein. In some embodiments of the methods, the detection molecule comprises a fluorescent molecule. In some embodiments of the methods, the detecting of step iv) comprises detecting a fluorescent signal from detection molecule, wherein the presence of the fluorescent signal from the detection molecule denotes the presence of SARS-CoV-2 in the sample. In some embodiments of the methods, the detecting of step iv) comprises detecting the presence of the detection molecule using an automated reader or a smartphone. In some embodiments of the methods, the solid support comprises a microplate. In some embodiments of the methods, the solid support comprises a microfluidic device. In some embodiments of the methods, the solid support comprises a bead. In some embodiments of the methods, the capture molecule binds SARS-CoV-2 alpha, beta, gamma, and delta spike protein variants. In some embodiments of the methods, the method further comprises treating the subject based on the detection of SARS-CoV-2 in the sample.

In a further aspect of the current disclosure, kits for detecting the presence of SARS-CoV-2 in a sample are provided. In some embodiments of the kits, the kits comprise: i) a capture molecule comprising a DNA nanostructure linked to one or more target-specific binding peptides, wherein the one or more target-specific binding peptides bind SARS-CoV-2, wherein the capture molecule is linked to a solid support; ii) a detection molecule comprising a SARS-CoV-2 specific antibody (SAB). In some embodiments of the kits, the kits further comprise a detection reagent. In some embodiments of the kits, the DNA nanostructure is linked to more than one target-specific binding peptides. In some embodiments of the kits, the DNA nanostructure is linked to three target-specific binding peptides. In some embodiments of the kits, one or more of the target-specific binding peptides are LCB1. In some embodiments of the kits, the SAB binds to the N-terminal domain of the SARS-CoV-2 spike protein. In some embodiments of the kits, the solid support comprises a microplate. In some embodiments of the kits, the solid support comprises a microfluidic device. In some embodiments of the kits, the solid support comprises a bead. In some embodiments of the kits, the capture molecule binds SARS-CoV-2 alpha, beta, gamma, and delta spike protein variants.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1: Overview of approach. Panel A) shows nanobodies bind to a target (the SARS-CoV-2 spike protein trimer here) through three CDR loops (yellow, blue, green), but must target the key ACE2 binding interface to block infection. Panel B) shows a programmable DNA nanostructure that positions three protein or peptide ligands can block any protein without having to bind to the key interface directly.

FIG. 2: Protein bioconjugation chemistry and preliminary data. Proteins can be conjugated to DNA (red sphere) using either cysteine based chemistry shown in Panel (A) or copper-free click chemistry with 4-azidophenylalanine-containing proteins shown in Panel (B). Panel C) shows MALDI-TOF mass spectrum of a peptide-DNA conjugate with an RBD-binding sequence P1. The DNA sequence is SEQ ID NO: 4 and the P1 protein sequence is SEQ ID NO: 5. Panel D) shows ELISA assay: LCB1 protein was immobilized on a surface and exposed to increasing concentrations of the spike S1 (RBD-containing) protein, followed by a primary antibody and a secondary antibody-HRP conjugate. Competition with excess free LCB1 abrogated the interaction. Panel E) shows surface plasmon resonance (SPR) analysis of monomeric spike RBD on a surface exposed to LCB1 in solution.

FIG. 3: Nanostructures used. Panel A) shows triangular DNA origami with handles for capturing a homotrivalent protein-DNA conjugate in the central cavity shown in panel (B). Panel C) shows native PAGE of three different DNA nanostructures: a 3-way junction, 6-helix bundle, and tetrahedral cage. All three can be annealed at high yield and purify. Panel D) shows hybrid protein-DNA cage from protein-DNA conjugates. Panel E) shows block-like DNA origami cuboid with addressable faces. F) Reconfiguarable DNA nano-tweezer with tunable arm lengths and distances between them. Panels (G-L) show proposed nano-scaffold designs for DNA-peptide hybrid molecules: four-helix bundle (G), six-helix bundle (H), two variants of a three-way junction (I, J), tetrahedral cage (K), and three-legged “stool” (L). All structures are scaled to roughly the same dimensions (with 5 nm scale indicated). Proteins and peptides can be attached to the ends of all helices, as well as nick points in the sides of the tetrahedral cages (indicated by red asterisks in (K)).

FIG. 4: In silico nanostructure evolution. A starting design, with its three peptide attachment sites colored in red, green and blue, is mutated by introducing a single-stranded region (pink) with 3 Tbases. The mean structures obtained from oxDNA simulation show that the distances between the peptide-functionalized sites change by several nanometers.

FIG. 5: Homo-trivalent LCB1 DNA-peptide hybrid molecules. A) To-scale model of a 4-helix bundle with three copies of LCB1 binding to the spike protein trimer (4HB-LCB1). B) AFM image of 4HB-LCB1. C) Inhibition of spike protein monomer binding to immobilized LCB1; both free LCB1 and its ssDNA conjugate inhibit equally. D) The trivalent 4HB-LCB1 DNA-peptide hybrid molecule is more effective at inhibiting RBD binding than monomeric LCB1. E) Heat map showing how far (blue: closer, red: farther) one arm (red arrow) of a DNA-peptide hybrid molecule can reach when another arm (green arrow) is bound to a known site. F) Computationally predicted binding sites for the nanobody that targets the spike NTD. Red arrows indicate spurious predictions, the green arrow indicates the known, correct NTD site.

FIG. 6: DNA-peptide hybrid molecule photocleavage. Attaching the protein ligands to the DNA-peptide hybrid molecule via DNA with photocleavable linkers will allow for removal of the nanostructure—and restoration of the protein-protein interaction—upon exposure to UV light.

FIG. 7: “CLASP” system. Panel A) shows standard IgG antibody structure with variable heavy (VH) region comprised of three CDRs. Panel B) shows CDR3 native conformation compared to the cyclized constrained CDR 3 peptide (the CLASP system). “B” denotes a bioconjugation handle, e.g. an alkyne for click.

FIG. 8: Validation of temporally sensitive TBI CLASPs. Panels A-D show qualitative representation of acute TBI CLASP (green) and cell nuclei (blue) on 1 dpi mouse CCI tissue (A, B), sham control mouse tissue (C) or 7 dpi CCI tissue (D). Panel E shows subacute TBI CLASP staining on 7 dpi mouse CCI tissue.

FIG. 9: Likely binding sites for the known fibrinogen (PDB: 1fza) binding peptide GPRPXX (SEQ ID NO: 3) obtained from global docking software GalaxyPepdock. Nanorulers will be designed to connect candidate sites for CD3 peptides from phage display experiments and this validated GPRPXX (SEQ ID NO: 3) binding pocket.

FIG. 10: Schematics of the DNA linked immunosorbent signal amplification assay (DLISA): the nanobody is coated on a plate as a capture domain, which binds with spike N-term binding domain (NTD); The mini-binder conjugated with DNA-fluorophore as an amplified signal probe that enhances the sensitivity and specificity in the identification of SARS-CoV-2 variants of concern (VOC).

FIG. 11: Schematics of the DLISA fast-simple detection. Panel A) shows the principle of DNA linked immunosorbent signal amplification assay (DLISA). Panel B) shows microfluidic digital DLISA. Panel C) shows a smartphone DLISA.

FIG. 12: Panels A-D) show LCB binding with spike trimer equivalent constants by SPR.

FIG. 13: SPR single cycle kinetics of mini binder & spike interaction. Panel A) shows Sensorgram of the response (RU) versus time of the single cycle kinetics assay performed by injecting concentration of 0.1 μM of mini binder on the spike wild type substrate. Panel B) shows the spike alpha variant substrate. Panel D) shows the spike gamma variant substrate. Panel C) shows the spike delta variant substrate

FIG. 14: Traditional Enzyme-Linked Immunoassay (ELISA) sandwich assay versus DNA Link Immuno Signal Amplification (DLISA) for SARS-CoV-2 diagnosis.

FIG. 15: The workflow of the imaging-based digital DLISA. A drop of patient fluidic sample is injected into microfluidic channel from the inlet and incubated in zones 1-4 sequentially for target capture and generation of a concentration gradient of the viral particle. After immunoreaction, a gradient of the nanobody, viral particle, and minibinder probe immuno-complex is formed along the channel bottom. Raw fluorescence images are collected at each zone under a microscope using a camera. The raw image is processed to digitally count the number of immunocomplexes. The concentrations of viral particle in patient samples are calculated by the gradient-based standard curve (concentration vs particle counts. The particle counts of zone 1 minus zone 4 is taken to subtract the non-specific binding effect).

FIG. 16: A schematic representation of the SPR principle.

FIG. 17: Primer exchange reaction cascades. Panel A shows a schematic for autonomous stepwise growth of a primer. Panel B shows a gel depicting a reaction of PER in different condition.

FIG. 18: Protein bioconjugation chemistry. Panel A shows protein can be conjugated to DNA using cysteine-based chemistry. Panel B shows gel depicting a mini-binder conjugate with DNA. Panel C shows the specificity of mini binder was evaluated by ELISA compared with BSA protein.

FIG. 19: Schematic illustration of imaging-based digital DLISA assay setup. A sandwich immunoassay will be carried in a microfluidic chip and imaged with an inverted fluorescence microscope. The inner bottom (glass) of the microfluidic chip is modified with capture nanobody via epoxysilane coupling chemistry. SARS-CoV-2 viral particle is captured on the surface and detected via the fluorescence signal generated from the minibinder probes bound to the viral surface. The microfluidic channel can be divided into multiple zones to generate a gradient of binding signal, which can be used to subtract the non-specific bindings created by the complex components in human fluidic sample if needed.

### DETAILED DESCRIPTION

In one aspect of the current disclosure, a method of detecting the presence of SARS-CoV-2 in a sample from a subject is provided. In some embodiments, the method comprises: i) contacting the sample to a capture molecule, wherein the capture molecule is linked to a solid support; contacting the sample to a capture molecule, the capture molecule comprising a nanobody specific for SARS-CoV-2, wherein the capture molecule is linked to a solid support; ii) incubating the sample in the presence of the capture molecule under conditions for SARS-CoV-2 in the sample to bind to the capture molecule, thereby forming a “V-AB” complex; iii) contacting the V-AB complex with a detection molecule under conditions to allow the detection molecule to bind the V-AB complex, the detection molecule comprising a DNA-peptide hybrid molecule, the DNA-peptide hybrid molecule comprising a DNA nanostructure chemically linked to one or more target-specific binding peptides, wherein the target-specific binding peptides specifically binds SARS-CoV-2; iv) detecting the presence of SARS-CoV-2 in the sample based on the presence of the bound detection molecule.

In some embodiments, the compositions disclosed herein are used in methods of detecting, purifying, or isolating a target of interest. In some embodiments, the methods comprise contacting a sample containing the target of interest to the DNA-peptide hybrid molecules, wherein the target-specific binding peptide(s) bind one or more sites on the target of interest. In some embodiments, the methods disclosed herein are useful to detect the presence of a target molecule of interest in a sample from a subject. In some embodiments, the methods disclosed herein are used to detect the presence of SARS-CoV-2 in a sample from a subject.

As used herein, “detection molecule” is a molecule or composition that binds to a target that comprises a detectable marker. In some embodiments, exemplary detection molecules include DNA-peptide hybrid molecules comprising a DNA nanostructure chemically linked to one or more target-specific binding peptides, wherein the target-specific binding peptides specifically binds SARS-CoV-2 further comprising a detectable marker.

In some embodiments, the DNA-peptide hybrid molecules are linked to a solid support, or are linked to a detectable marker. In some embodiments, detectable markers include fluorescent molecules, for example, fluorescent proteins, quantum dots, fluorescein and derivatives thereof. In some embodiments, detectable markers include luminescent molecules, for example, luciferase. In some embodiments, detectable markers include molecules capable of converting a substrate in a recognizable manner, for example, horseradish peroxidase, alkaline phosphatase, glucose oxidase, and b-galactosidase or other such labels well-known in the art.

### Terminology

Unless defined otherwise, all technical and scientific terms used herein have the same meaning as commonly understood by one of skill in the art to which the invention pertains. All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

A range includes each individual member. Thus, for example, a group having 1-3 members refers to groups having 1, 2, or 3 members.

It should also be understood that, unless clearly indicated to the contrary, in any methods claimed herein that include more than one step or act, the order of the steps or acts of the method is not necessarily limited to the order in which the steps or acts of the method are recited.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein, “about,” “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the term “nanostructure” is a defined structure having at least one dimension (e.g., length, width, thickness) in the nanoscale range (approximately 1 nanometer (nm) to 100 nm).

The term “DNA nanostructure”, as used herein, refers to a nanostructure at least partially composed of DNA assembled in a defined structure and having at least one dimension (e.g., length, width, thickness) in the nanoscale range (approximately 1 nm to 100 nm). By way of example but not by way of limitations, DNA nanostructures include single-stranded DNA molecules, a three-helix bundle, a four-helix bundle, a six-helix bundle, a triangular DNA origami structure, a tetrahedral wireframe cage, a block-like origami cuboid, reconfigurable tweezers, double crossover tiles, branched three-way junctions, a three-legged stool. In some embodiments, a DNA nanostructure comprises one or more single-stranded DNA molecules. In some embodiments, the one or more single-stranded molecules are configured to initiate a primer exchange reaction. Methods of forming DNA nanostructures such as those exemplified in this disclosure, are known in the art, see e.g., list of cited references, which are incorporated herein by reference in their entireties.

As used herein, “DNA-peptide hybrid molecule” refers to a molecule comprises a DNA molecule chemically linked to a peptide molecule thereby generating a DNA-peptide hybrid molecule. In some embodiments, the DNA molecule comprises a DNA nanostructure.

In some embodiments, orthogonal chemical reactions are used to link DNA to peptides generating the DNA-peptide hybrid molecules of the current disclosure. In some embodiments, the chemical linkage is reversible. In some embodiments, the DNA-peptide hybrid molecule comprises one or more single-stranded nucleic acid molecules configured to initiate a primer exchange reaction.

As used herein, “detecting”, or grammatical variations thereof, refers to the process of identifying the presence or absence of a particular molecule. In some embodiments, detecting is performed without the use of a mental process by an individual and comprises an automatic process performed by a machine or instrument. In some embodiments, detection comprises measuring light emitted by a fluorescent molecule or other detectable moiety. Such moieties and methods for their detection are well-known in the art.

As used herein, “primer exchange reaction” or “PER” refers to a process which grows nascent single-stranded DNA with user-specified sequences following prescribed reaction pathways. Details surrounding the design and function of PER reagents can be found in Kishi J. Y. et al. Nature Chem. 10, 155-164 (2018), which is incorporated by reference herein in its entirety. In some embodiments, PER is used to amplify a suitable single-stranded DNA sequence which in some embodiments, comprises all or a portion of the DNA nanostructure. By way of example, amplification of the single-stranded member of the DNA nanostructure using PER to incorporate labeled nucleotides (e.g., fluorescently labeled nucleotides) into the growing DNA strand results in signal generation and/or signal amplification, which is, in some embodiments, used as the detection reagent for SARS-CoV-2 spike protein.

As used herein, “LCB1” refers to a peptide with the sequence DKEWILQKIYEIMRLLDELGHAEASMRVSDLIYEFMKKGDERLLEEAERLLEEVER (SEQ ID NO: 2). More information surrounding the properties and the use of LCB1 can be found in the publication Cao L et al. Science. VI. 370, No. 6515 pp. 426-431, 2020, incorporated by reference herein in its entirety. In some embodiments, LCB1 is chemically linked to a DNA nanostructure. In some embodiments, more than one LCB1 molecule is chemically linked to one DNA nanostructure.

The phrases “% sequence identity,” “percent identity,” or “% identity” refer to the percentage of amino acid residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated by reference herein in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other compositions described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, etc.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O (6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “nucleic acid handle”, as used herein, is a nucleic acid attached to or intended for attachment to a polypeptide and having at least some nucleic acid bases available for hybridization to complementary nucleic acid strands of a nucleic acid mold or other structure. Nucleic acid handles may include single-stranded DNA, double-stranded DNA with at least a portion of single-stranded DNA, RNA, aptamers, and peptide nucleic acids (PNAs), or combinations thereof.

The phrase “DNA origami nanostructure” as used herein refers to a nanostructure composed of DNA folded into a precise two- or three-dimensional shape. A DNA origami nanostructure as described herein may function as a DNA mold.

As used herein, “orthogonal chemical reactions” refers to different chemical reactions that occur selectively and in high yield in the presence of other functional groups. Exemplary orthogonal reactions include, but are not limited to, click chemistry (“click reaction”), maleimide chemistry, disulfide formation, oxime formation between an aminooxy group and a ketone/aldehyde, tetrazine/trans-cyclooctene conjugation, enzymatic ligations (e.g., transglutaminase), copper-catalyzed click reactions, and tyrosine oxidation reactions. Various other reactions may include those described in Stephanopoulos, N., “Hybrid Nanostructures from the Self-Assembly of Proteins and DNA”, Chem. 6, pp. 364-405, 2020, incorporated by reference herein in its entirety.

As used herein, the term “click reaction” refers to the reaction of an azide group with an alkyne group to form a 5-membered heteroatom ring.

As used herein, “target-specific binding peptide” is a polypeptide molecule that is able to bind to another protein, peptide, or other molecule of interest. Target-specific binding peptides may be chemically linked, for example, to DNA nanostructures. In some embodiments, more than one target-specific binding peptides are linked to a single DNA nanostructure. In some embodiments, linking more than one target-specific binding peptides to one DNA nanostructure increases the affinity of the DNA nanostructure-peptide hybrid compared to the target-specific binding protein alone. In some embodiments, the peptide LCB1 is a target-specific binding peptide. As used herein, “target-specific” refers to the property of a molecule having a high affinity for another molecule. In some embodiments, target specific molecules may have a K_{d} or dissociation constant of less than 1 micromolar, or preferably less than 5 nanomolar with a target molecule. In some embodiments, a target-specific binding peptide comprises an antibody or portion thereof, such as a nanobody. In some embodiments, a target-specific binding protein comprises a mini-binder, such as LCB1.

As used herein, “capture molecules” are molecules or compounds that bind to SARS-CoV-2 that are, in some embodiments, linked to a solid support. In some embodiments, capture molecules are antibodies, Fabs, or nanobodies. Exemplary nanobodies for use in the methods and kits of the current disclosure can be found in the reference Schoof, M. et al. An ultrapotent synthetic nanobody neutralizes SARS-CoV-2 by stabilizing inactive Spike. Science 370, 1473-1479, doi: 10.1126/science.abe3255 (2020), which is incorporated by reference herein in its entirety. In some embodiments, capture molecules are DNA-peptide hybrid molecules that bind to SARS-CoV-2 surface glycoprotein.

As used herein, “photocleavable linkage” is a chemical link between two or more molecules that can be cleaved upon exposure to light of a given wavelength or energy. In some embodiments, o-nitrobenzyl ester moieties are installed into the DNA backbone of a DNA-peptide hybrid molecule such that, upon exposure to 350 nm ultraviolet (UV) light, the chemical linkages in the DNA molecule are cleaved. In some embodiments, placement of the cleavable linkages is selected such that the cleavage separates the DNA portion of the molecule from the peptide portion of the molecule. Therefore, in the context of a DNA-peptide hybrid molecule, wherein the peptide portion of the molecule binds specifically to a target molecule, the cleavage of the o-nitrobenzyl ester moieties in the DNA portion of the molecule upon exposure to 350 nm UV light effectively separates the target-binding, i.e., peptide portion of the molecule, from the rest of the molecule.

As used herein, “binding affinity” or “affinity” is the strength of the binding interaction between a single molecule and its ligand or binding partner.

As used herein, “binding avidity”, “avidity”, or “functional affinity” is the strength of binding between a molecule comprising multiple target-binding sites and the target molecule. In some embodiments, the DNA-peptide hybrid molecules of the present disclosure comprise multiple target-specific peptides bound to a single DNA nanostructure. Therefore, the avidity of the DNA-peptide hybrid molecule is the strength of the binding of the complete structure of the molecule including the multiple target-specific binding peptides to the target molecule.

As used herein, “immunoglobulin Fc domain” or “Fc domain” refers to the fragment crystallizable domain or the tail region of an antibody that interacts with cell surface receptors called Fc receptors and some proteins of the complement system. This property allows antibodies to activate the immune system. In IgG, IgA and IgD antibody isotypes, the Fc region is composed of two identical protein fragments, derived from the second and third constant domains of the antibody's two heavy chains; IgM and IgE Fc regions contain three heavy chain constant domains (CH domains 2-4) in each polypeptide chain. In some embodiments, the DNA-peptide hybrid molecules of the present disclosure comprise an immunoglobulin Fc domain. In some embodiments, the type of Fc domain selected is designed such that the appropriate immune response is instigated by the Fc domain selected. For example, the properties of the Fc domains are known in the art and include the ability to promote antibody directed cellular cytotoxicity (ADCC). As used herein, “antibody directed cellular cytotoxicity” or “ADCC” refers to lysis of target cells coated with antibody by effector cells with cytolytic activity and specific immunoglobulin receptors called Fc receptors, including NK cells, macrophages, and granulocytes.

As used herein, “nanobody” refers to a single monomeric variable antibody domain, also known as single-domain antibodies (sdAbs) that are able to bind selectively to a specific antigen.

As used herein, “antigen” refers to a molecule that is capable of stimulating the immune system of a subject.

As used herein, “paratope” refers to region of an antibody that binds to the antigen-binding site (epitope) of the target molecule.

In some embodiments, the DNA-peptide hybrid molecules of the present disclosure which, in some embodiments, are designed to bind to a target molecule, can be “sized” or “tuned” to match the distance and/or arrangement of the binding domains in the target molecule. Put another way, if, for example, the target molecule contains two target-binding domains for which the DNA-hybrid molecule is designed to bind, that are 5 nm apart, the DNA nanostructure may be sized or tuned such that the target-specific binding peptides, when attached to the DNA nanostructure, are located about 5 nm apart in a conformation that enables favorable access of the target-specific binding peptides to the target-binding domains. Thus, without being limited by any theory or mechanism, this tunable property of the compositions of the current disclosure is thought to enable rational design of DNA nanostructures that takes advantage of the property of avidity of multiple binding domains binding to a single target molecule. In essence, being able to be tuned increases the functional affinity of the DNA-hybrid molecule to its target molecule when compared to the affinity of a similar molecule that does not present the target-specific binding peptides in a conformation that allows them to be accessible to the target binding regions of the target molecule.

As used herein, “infectious disease” refers to diseases caused by pathogenic microorganisms including, for example, bacteria, fungi, viruses and eukaryotic parasites. In some embodiments, the infectious disease is coronavirus disease discovered in 2019 (COVID-19) caused by severe acute respiratory syndrome coronavirus 2 (SARS-CoV-2).

As used herein, “autoimmune disease” refers to a disease or disorder wherein a subject's immune system attacks normal cells and tissues in the subject.

As used herein, “cancer” refers to a large group of cell proliferative disorders caused by an uncontrolled division of abnormal cells.

As used herein, “psychiatric disease or disorder” refers to wide variety of behavioral or mental patterns that cause significant distress or impairment of personal functioning in affected subjects. Psychiatric diseases or disorders are caused by abnormal functioning of the central nervous system.

As used herein, “environmental exposure” refers to contact with chemical, biological, or physical substances found in air, water, food, or soil that may have a harmful effect on a person's health.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

| SARS-COV-2 surface glycoprotein or “spike” protein sequence: | |
|---|---|
| (SEQ ID NO: 1) | |
| MFVFLVLLPL VSSQCVNLTT RTQLPPAYTN SFTRGVYYPD KVFRSSVLHS TQDLFLPFFS | 60 |
| NVTWFHAIHV SGTNGTKRFD NPVLPFNDGV YFASTEKSNI IRGWIFGTTL DSKTQSLLIV | 120 |
| NNATNVVIKV CEFQFCNDPF LGVYYHKNNK SWMESEFRVY SSANNCTFEY VSQPFLMDLE | 180 |
| GKQGNFKNLR EFVFKNIDGY FKIYSKHTPI NLVRDLPQGF SALEPLVDLP IGINITRFQT | 240 |
| LLALHRSYLT PGDSSSGWTA GAAAYYVGYL QPRTELLKYN ENGTITDAVD CALDPLSETK | 300 |
| CTLKSFTVEK GIYQTSNFRV QPTESIVRFP NITNLCPFGE VFNATRFASV YAWNRKRISN | 360 |
| CVADYSVLYN SASFSTFKCY GVSPTKLNDL CFTNVYADSF VIRGDEVRQI APGQTGKIAD | 420 |
| YNYKLPDDFT GCVIAWNSNN LDSKVGGNYN YLYRLFRKSN LKPFERDIST EIYQAGSTPC | 480 |
| NGVEGFNCYF PLOSYGFQPT NGVGYQPYRV VVLSFELLHA PATVCGPKKS TNLVKNKCVN | 540 |
| FNFNGLTGTG VLTESNKKFL PFQQFGRDIA DTTDAVRDPQ TLEILDITPC SFGGVSVITP | 600 |
| GTNTSNQVAV LYQDVNCTEV PVAIHADQLT PTWRVYSTGS NVFQTRAGCL IGAEHVNNSY | 660 |
| ECDIPIGAGI CASYQTQTNS PRRARSVASQ SIIAYTMSLG AENSVAYSNN SIAIPTNFTI | 720 |
| SVTTEILPVS MTKTSVDCTM YICGDSTECS NLLLQYGSFC TQLNRALTGI AVEQDKNTQE | 780 |
| VFAQVKQIYK TPPIKDFGGF NFSQILPDPS KPSKRSFIED LLFNKVTLAD AGFIKQYGDC | 840 |
| LGDIAARDLI CAQKFNGLTV LPPLLTDEMI AQYTSALLAG TITSGWTFGA GAALQIPFAM | 900 |
| QMAYRFNGIG VTQNVLYENQ KLIANQFNSA IGKIQDSLSS TASALGKLQD VVNQNAQALN | 960 |
| TLVKQLSSNF GAISSVLNDI LSRLDKVEAE VQIDRLITGR LQSLQTYVTQ QLIRAAEIRA | 1020 |
| SANLAATKMS ECVLGQSKRV DFCGKGYHLM SFPQSAPHGV VFLHVTYVPA QEKNFTTAPA | 1080 |
| ICHDGKAHFP REGVFVSNGT HWFVTQRNFY EPQIITTDNT FVSGNCDVVI GIVNNTVYDP | 1140 |
| LOPELDSFKE ELDKYFKNHT SPDVDLGDIS GINASVVNIQ KEIDRLNEVA KNLNESLIDL | 1200 |
| QELGKYEQYI KWPWYIWLGF IAGLIAIVMV TIMLCCMTSC CSCLKGCCSC GSCCKFDEDD | 1260 |
| SEPVLKGVKL HYT | |

The “alpha” variant of SARS-CoV-2, or B.1.1.7 variant has the following mutations: 69-70del, N501Y, and P681H.

The “beta” variant of SARS-CoV-2, or B.1.351 variant has the following mutations: K417N, E484K and N501Y.

The “gamma” variant of SARS-CoV-2, or P.1 variant has the following mutations: K417T, E484K, and N501Y.

The “delta” variant of SARS-CoV-2, or B.1.617.2 variant has the following mutations: L451R, T478K, and P681R.

### EXEMPLARY EMBODIMENTS

1. A method of detecting the presence of SARS-CoV-2 in a sample from a subject, the method including:
i) contacting the sample to a capture molecule, the capture molecule including a nanobody specific for SARS-CoV-2, wherein the capture molecule is linked to a solid support;
ii) incubating the sample in the presence of the capture molecule under conditions for SARS-CoV-2 in the sample to bind to the capture molecule, thereby forming a “V-AB” complex;
iii) contacting the V-AB complex with a detection molecule under conditions to allow the detection molecule to bind the V-AB complex, the detection molecule including a DNA-peptide hybrid molecule, the DNA-peptide hybrid molecule including a DNA nanostructure chemically linked to one or more target-specific binding peptides, wherein the target-specific binding peptides specifically binds SARS-CoV-2;
iv) detecting the presence of SARS-CoV-2 in the sample based on the presence of the bound detection molecule.

2. The method of embodiment 1, wherein the DNA nanostructure of the detection molecule includes one of: a single-stranded DNA molecule, a three-helix bundle, a four-helix bundle, a six-helix bundle, a triangular DNA origami structure, a tetrahedral wireframe cage, a block-like origami cuboid, reconfigurable tweezers, double crossover tiles, branched three-way junctions, and a three-legged stool.

3. The method of embodiments 1 or 2, wherein the DNA nanostructure is linked to more than one target-specific binding peptide.

4. The method of any of the preceding embodiments, wherein the DNA nanostructure is linked to three target-specific binding peptides.

5. The method of any of the preceding embodiments, wherein one or more of the target-specific binding peptides includes LCB1.

6. The method of any of the preceding embodiments, wherein the capture molecule includes a nanobody that specifically binds to the N-terminal domain of the SARS-CoV-2 spike protein.

7. The method of any of the preceding embodiments, wherein the detection molecule includes a fluorescent molecule

8. The method of embodiment 7, wherein the detecting of step iv) includes detecting a fluorescent signal from the detection molecule, wherein the presence of the fluorescent signal from the detection molecule denotes the presence of SARS-CoV-2 in the sample.

9. The method of embodiment 7 or 8, wherein the detecting of step iv) includes detecting the presence of the detection molecule using an automated reader or a smartphone.

10. The method of any of the preceding embodiments, wherein the solid support includes a microplate.

11. The method of any of the preceding embodiments, wherein the solid support includes a microfluidic device.

12. The method of any of the preceding embodiments, wherein the solid support includes a bead.

13. The method of any of the preceding embodiments, wherein the DNA nanostructure includes a single stranded DNA molecule.

14. The method of embodiment 13, wherein the single-stranded DNA molecule include fluorescent labels.

15. The method of embodiment 13, wherein detection includes a primer exchange reaction (PER).

16. The method of embodiment 15, further including contacting the detection molecule with fluorescently labeled oligonucleotides that hybridize with the product of the PER.

17. The method of any of the preceding embodiments, wherein the target-specific binding peptide binds SARS-CoV-2 alpha, beta, gamma, and delta spike protein variants.

18. The method of any of the preceding embodiments, wherein the method further includes treating the subject based on the detection of SARS-CoV-2 in the sample.

19. A kit for detecting the presence of SARS-CoV-2 in a sample including:
i) a capture molecule linked to a solid support, wherein the capture molecule is a nanobody specific for SARS-CoV-2;
ii) a detection molecule including a DNA nanostructure linked to one or more target-specific binding peptides, wherein the one or more target-specific binding peptides bind SARS-CoV-2.

20. The kit of embodiment 16, further including a detection reagent.

21 The kit of embodiments 19 or 20, wherein the DNA nanostructure is linked to more than one target-specific binding peptides.

22. The kit of any of embodiments 19-21, wherein the DNA nanostructure is linked to three target-specific binding peptides.

23. The kit of any of embodiments 19-22, wherein one or more of the target-specific binding peptides are LCB1.

24. The kit of any of embodiments 19-23, wherein the DNA nanostructure includes a fluorescent molecule.

25. The kit of any of embodiments 19-24, wherein the capture molecule binds to the N-terminal domain of the SARS-CoV-2 spike protein.

26. The kit of any of embodiments 19-25, wherein the solid support includes a microplate.

27. The kit of any of embodiments 19-26, wherein the solid support includes microfluidic device.

28 The kit of any of embodiments 19-27, wherein the solid support includes a bead.

29. The kit of any of embodiments 19-28, wherein the DNA nanostructure includes a single-stranded DNA molecule.

30. The kit of embodiment 29, including components for a primer extension reaction, including DNA hairpin probes, and optionally, a polymerase, and labeled oligonucleotides.

31 The kit of any of embodiments 17-30, wherein the target-specific binding peptide binds SARS-CoV-2 alpha, beta, gamma, and delta spike protein variants.

32. A method of detecting the presence of SARS-CoV-2 in a sample from a subject, the method including:
i) contacting the sample to a capture molecule, the capture molecule including a DNA-peptide hybrid molecule, the DNA-peptide hybrid molecule including a DNA nanostructure chemically linked to one or more target-specific binding peptides, wherein the capture molecule is linked to a solid support;
ii) incubating the sample in the presence of the capture molecule under conditions for SARS-CoV-2 in the sample to bind to the capture molecule, thereby forming a “V-AB” complex;
iii) contacting the V-AB complex with a detection molecule under conditions to allow the detection molecule to bind the C-AB complex, the detection molecule including a SARS-CoV-2 specific binding molecule (SBM);
iv) detecting the presence of SARS-CoV-2 in the sample based on the presence of the bound detection molecule.

33 The method of embodiment 32, wherein the detection molecule includes a nanobody specific for SARS-CoV-2.

34 The method of embodiment 32 or 33, wherein the DNA nanostructure is selected from the group consisting of: a single-stranded DNA molecule, a three-helix bundle, a four-helix bundle, a six-helix bundle, a triangular DNA origami structure, a tetrahedral wireframe cage, a block-like origami cuboid, reconfigurable tweezers, double crossover tiles, branched three-way junctions, and a three-legged stool.

35. The method of any of embodiments 32-34, wherein the DNA nanostructure is linked to more than one target-specific binding peptide.

36. The method of any of embodiments 32-35, wherein the DNA nanostructure is linked to three target-specific binding peptides.

37. The method of any of embodiments 32-36, wherein one or more of the target specific binding peptides are LCB1.

38. The method of any of embodiments 32-38, wherein the capture molecule binds to the receptor binding domain of the SARS-CoV-2 spike protein.

39. The method of any of embodiments 32-38, wherein the detection molecule includes a fluorescent molecule.

40. The method of any of embodiments 32-39, wherein the detecting of step iv) includes detecting a fluorescent signal from detection molecule, wherein the presence of the fluorescent signal from the detection molecule denotes the presence of SARS-CoV-2 in the sample.

41. The method of any of embodiments 32-39, wherein the detecting of step iv) includes detecting the presence of the detection molecule using an automated reader or a smartphone.

42. The method of any of embodiments 32-41, wherein the solid support includes a microplate.

43. The method of any of embodiments 32-42, wherein the solid support includes a microfluidic device.

44. The method of any of embodiments 32-43, wherein the solid support includes a bead.

45. The method of any of embodiments 32-44, wherein the capture molecule binds SARS-CoV-2 alpha, beta, gamma, and delta spike protein variants.

46. The method of any of embodiments 32-45, wherein the method further includes treating the subject based on the detection of SARS-CoV-2 in the sample.

47. A kit for detecting the presence of SARS-CoV-2 in a sample including:
i) a capture molecule including a DNA nanostructure linked to one or more target-specific binding peptides, wherein the one or more target-specific binding peptides bind SARS-CoV-2, wherein the capture molecule is linked to a solid support;
ii) a detection molecule including a SARS-CoV-2 specific antibody (SAB).

48 The kit of embodiment 47, further including a detection reagent.

49. The kit of embodiments 47 or 48, wherein the DNA nanostructure is linked to more than one target-specific binding peptides.

50. The kit of any of embodiments 47-49, wherein the DNA nanostructure is linked to three target-specific binding peptides.

51. The kit of any of embodiments 47-50, wherein one or more of the target-specific binding peptides are LCB1.

52. The kit of any of embodiments 47-51, wherein the SAB binds to the N-terminal domain of the SARS-CoV-2 spike protein.

55. The kit of any of embodiments 47-54, wherein the solid support includes a microplate.

56. The kit of any of embodiments 47-55, wherein the solid support includes a microfluidic device.

57 The kit of any of embodiments 47-56, wherein the solid support includes a bead.

58. The kit of any of embodiments 47-57, wherein the capture molecule binds SARS-CoV-2 alpha, beta, gamma, and delta spike protein variants.

### EXAMPLES

### Example 1

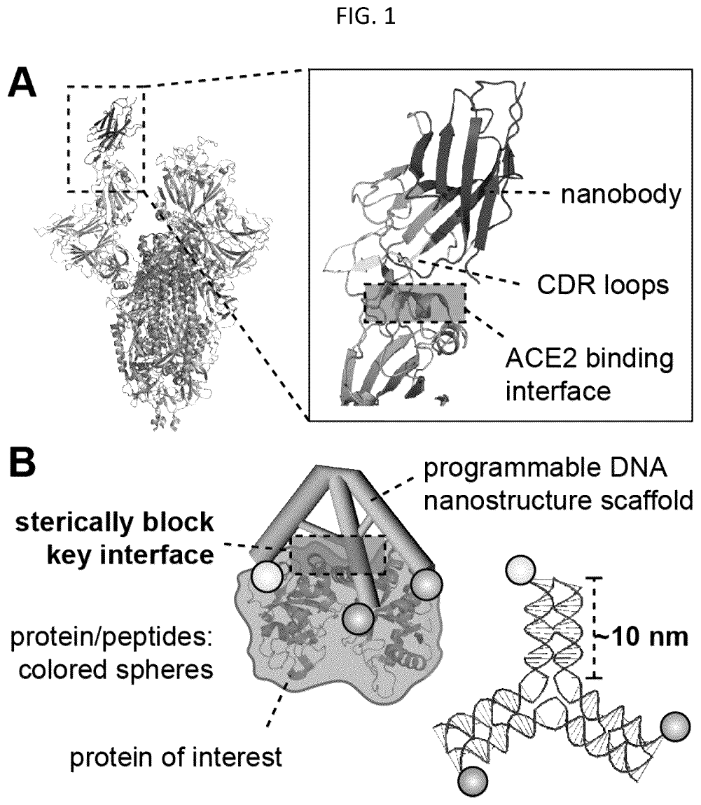

Blocking protein-protein interactions is crucial for biological studies. The ability to block protein-protein interactions (PPIs) is crucial not just for therapeutic purposes—e.g. neutralizing antibodies for pathogenic threats like SARS-CoV-2, or small molecule drugs for cancer therapy—but also for fundamental biological studies. Countless biological processes are mediated by protein-protein interactions, such as cell-cell interactions, signal transduction, cell-matrix interactions, immune system recognition, and many others, but it can be difficult to block these interactions with high affinity and specificity. Approaches like small molecule drugs, or peptides found through rational design or high-throughput evolutionary methods like phage, mRNA, or ribosome display are often hindered by lack of binding to the key protein-protein interface. Antibodies can block PPIs, but again must target a key interface (FIG. 1A). Furthermore, these methods are generally not reversible or triggerable, and cannot be switched “on” and “off” on-demand with simple triggers. Creating a nanostructure that can switch PPIs on in a stimulus-responsive fashion (especially using light) would enable basic biology studies in targets that are not amendable to traditional optogenetic approaches. Furthermore, PPIs can span a large range of sizes, and it can be especially difficult to block multivalent interactions, as in viruses. Disclosed herein is a protein-DNA nanostructure platform whose dimensions can be precisely tuned to “match” a protein target, enveloping it and blocking its function (FIG. 1B). This approach does not rely on selectively binding the key interface; rather, Inventors propose to use a DNA nanostructure scaffold to position multiple peptides or proteins in 3D space to bind to different patches of the protein target, with the remainder of the structure sterically occluding the binding interface. The ability to reverse the nanostructure assembly (using precise stimuli like light) will impart spatiotemporal control to blocking the interaction.

Multivalent binding enhances affinity and expands target scope. One way to dramatically increase affinity for a target is by leveraging avidity: positioning multiple binding groups so that they can act cooperatively. Antibodies like IgG and IgM are intrinsically multivalent, although their geometry cannot be tuned to match the target. Extensive work in bionanotechnology has sought to rationally design multivalent binding agents for biomaterial applications. Most of these examples simply rely on a high density of the binding agents for activity, but a number of recent efforts have focused on matching the target size and valency with greater precision. For example, intrinsically symmetric assemblies can be targeted with designed homo-oligomeric binding agents. One report described a de novo designed homotrimeric protein grafted with a complementarity determining region (CDR) loop derived from a hemagglutinin (HA)-binding antibody could neutralize influenza with an IC₅₀ in the picomolar range. Precisely matching the HA trimer geometry (namely the distance between monomers and the threefold rotational symmetry) was critical to the binding affinity. Using a similar design, another group reported a starshaped DNA nanostructure that positioned aptamers to match the distance and fivefold symmetry of the dengue virus coat proteins, resulting in potent binding and virus inhibition (EC₅₀=2 nM). Once again, the ability to recapitulate the geometry of the capsid proteins with the aptamer was critical; structures that were too large or too small, or that had fewer or more than five ligands, did not bind as effectively. Furthermore, this example demonstrated the great potential of DNA as a programmable scaffold with controllable dimensions, relying on the precise valence and distances of the star-shaped scaffold to recapitulate the capsid protein symmetry.

The above examples, however, are restricted to homo-oligomeric targets like HA or viral capsids. Extending this paradigm to multiple different targets (either on a single protein or a protein complex) would dramatically expand the range of possible targets. Even simple DNA duplexes can be used as “molecular rulers” to position two binding groups, such as peptides or scFv molecules with a tunable distance to bind two separate sites on a target and enhance binding. In the latter example, two scFv fragments (either identical or different) could target HIV-1 virion spike proteins and improve virus neutralization by over 100-fold, whereas native IgG molecules were too large to effectively bind. Recent work also demonstrated that a DNA tile bearing two aptamer loops could be evolved to target non-overlapping sites of a target protein with femtomolar affinity, with the tile imparting the appropriate spacing to match the protein size. Here, inventors ask the question: can a DNA nanostructure be designed to position multiple protein binding groups with precise spatial control, but without the scaffold size limitations of antibodies or antibody mimetics? Such a general method that can position multiple (2-3) protein/peptide-based ligands, on a size- and shape-programmable scaffold is still lacking. These nanoscale synthetic antibodies, hereinafter “DNA-peptide hybrid molecules,” will be designed and optimized/“evolved” in silico using coarse-grained molecular dynamics simulations, in a feedback loop with experimental results.

DNA nano-scaffolds possess several key advantages over other display methods. The use of DNA nanostructures—such as DNA origami, multi-helical bundles, branched tiles, wireframe cages, or single stranded “brick” assemblies—to display peptides or proteins in a multivalent fashion has certain key benefits over other scaffolds like proteins, polymers, or self-assembled nanoparticles/fibers. These advantages include: (1) Facile presentation of multiple polypeptides (either identical or different) with stoichiometric control and user-defined valency; (2) Control over the spacing of the peptides with ˜3-5 nm resolution; (3) User-defined size and shape of the ultimate structure to best match a target size (up to tens of nanometers); (4) Attachment of the final targeting assembly with other nanoscale carriers like liposomes or nanoparticles via DNA hybridization; (5) Potential for multivalent, or bi-/multi-specific structures by oligomerizing individual DNA-peptide hybrid molecules using DNA; (6) Steric blockage of protein-protein interactions due to their large size; (7) Demonstrated stability and functionality in vivo of either bare nanostructures or after stabilization using simple peptide coatings; (8) Large scale (˜$100/gram) production using recent breakthrough DNA production methods; (9) Dynamic assembly/disassembly of structures using light18 or input displacement strands19. (10) Ability to be shielded from the immune system, or to stimulate an immune response depending on the desired application. (11) Capacity for intracellular delivery and subcellular trafficking. (12) Potential to target assembled protein complexes by combining binders to distinct components of the complex on a DNA scaffold. Inventors also highlight that using a rigid DNA nanostructure (as opposed to a simple dsDNA molecular ruler) will enable enhanced binding due to lower entropic penalties, and the use of three or more binding peptides/proteins with precise display in 3D space.

One aspect of the disclosed technology is to use a DNA nano-scaffold to control the spatial orientation of multiple binding peptides or proteins, to create a highly specific synthetic blocking agent for protein-protein interactions. In antibodies, a large portion of the sequence is dedicated to positioning a few key CDR loops in the correct conformation; the inventors' work effectively decouple this structural component from the binding agents. Unlike antibodies, however, our structures will be designed to match the given target size and geometry. This will enable not only tighter binding (even if the individual peptides/proteins have only modest affinity), but also blocking of the target cell surface receptors due to the steric bulk provided by the scaffolding nanostructure. Crucially, this method enables peptides that bind to areas away from the targeted interface to be converted to a blocking function through the appended nanoscaffold. Because our approach can use both short, synthetic peptides and larger, folded proteins, it serves as a rapid way to quickly extend binding agents found from other approaches (e.g. phage/mRNA/yeast/ribosome display, de novo designed proteins, or novel nanobodies or scFv fragments) to multivalent scaffolds. In addition to using reported peptide/proteins and designing nanostructures to best bind a target, inventors will also find novel binding agents for fibrin/fibrinogen, and attach them to a DNA scaffold in a multivalent fashion. All of these approaches include seamless molecular integration of the protein/peptide groups with a DNA nanoscaffold, with control over the linker length and rigidity, so tailored protein-DNA bioconjugation will play a role in these studies.

Another aspect of the disclosed technology is the in silico screening and optimization of hybrid peptide/protein-DNA nanostructures. Aspects to consider when designing nanostructures of the present disclosure include, but are not limited to: (1) enough rigidity so that there is no entropic penalty to binding, yet (2) sufficient flexibility to tolerate thermal fluctuations and imperfections in the design. To tune these competing forces, inventors develop the first integrated, coarse-grained model of protein-DNA nanostructures, where both molecules can be parameterized in a way that is accurate and computationally tractable. The model will in turn allow us to computationally screen multiple different DNA nanostructure designs, both in terms of geometry and strategic introduction of flexible/bulged sections, and to test the effect of peptide-DNA linker length and flexibility. Inventors will also employ computational models to best estimate pairwise distances between two binding agents whose binding site is unknown, and then use these distances as guidelines to design high-affinity blocking agents.

Currently, the major obstacle of in silico design in therapeutics are the system sizes and timescales involved in studying the binding pathways, as well as the correct parametrization of the models that predict binding interactions. As DNA nanostructures contain hundreds to several thousands of nucleotides, they are not amenable to atomistic-resolution computational studies that would sample their binding pathways to proteins. However, the coarse-grained approach allows for efficient sampling, making in silico evolutionary design possible by automatically generating and testing in simulation the binding of libraries of DNA nanostructures. Thus, this work will develop a new efficient design framework for automated evolutionary design, analysis and optimization of peptide/protein-DNA nanoscaffolds. Such a platform can greatly reduce experimental costs and speed-up development of high-affinity blocking DNA-peptide hybrid molecules. Although our work will develop and validate the system on the SARS-CoV-2 spike protein and fibrinogen as model systems, the emphasis will be on a workflow that can be readily adapted to new targets and new binding agents.

Overview: The overall goal was to create a method for designing DNA nanostructures that can spatially display 2-3 binding ligands (primarily peptides and proteins, though aptamers can also be employed) that bind to different portions of a given protein target. Accomplishing this goal, however, includes accurate methods for computationally modeling the hybrid protein/peptide-DNA nanostructure, and “docking” it with the target without too great of an entropic cost. Inventors will describe an integrated computational-experimental pipeline, where coarse-grained simulation methods will be used to design an initial set of DNA-peptide hybrid molecules that can be experimentally tested for binding. The results of these experiments will be used to refine the models and generate a library in silico of slightly mutated nanostructures, the best-performing of which will be selected for future rounds of experimental characterization.

In a first aspect, Inventors will focus on a target for which multiple binding groups are known—the SARS-CoV-2 spike protein receptor binding domain (RBD)—as a test bed in order to develop and benchmark the method. Inventors will create DNA-peptide hybrid molecules with three identical binding groups that target the known ACE2 binding site of the RBD. Inventors will then use one of these binding agents in conjunction with recently reported molecules that bind to a different region of the spike protein to develop hetero-bivalent structures. This process will involve novel chemical strategies for integrating the proteins/peptides with the DNA scaffold, optimizing the computational methods used, and testing DNA-peptide hybrid molecule “activity” by blocking the RBD interaction with the ACE2 receptor in a reversible fashion. In a second aspect, Inventors will use phage display to find several new nanobodies for fibrinogen, and then use these to discover heterobi- and tri-valent DNA-peptide hybrid molecules that bind to this target and block its activity in a stimulus-responsive, light-switchable fashion.

Develop DNA-peptide hybrid molecules for blocking the SARS-CoV-2 spike protein. The COVID-19 pandemic has highlighted the need for high-affinity binding/blocking agents for viral threats like SARS-CoV-2. As a result, there are a number of promising protein and peptide ligands for the spike trimer receptor binding domain (RBD), which is presented as a homotrimer with a known crystal structure on the capsid surface. Designing a DNA-peptide hybrid molecule that positions three identical proteins/peptides with a geometry and distances that match the RBD trimer will serve as an ideal test bed for both DNA nanostructure synthesis, but also to validate and optimize the theoretical model and computational pipeline. By the end of this aspect, Inventors will have demonstrated that a DNA scaffold bearing three identical protein/peptide binding groups can serve as a high-affinity blocking agent for a virus.

Synthesize RBD-binding proteins and peptides and conjugate them to DNA. Several protein/peptides have been reported that target the SARS-CoV-2 spike protein RBD and can neutralize virus association with the target ACE2 receptor. In particular, Inventors will explore three categories of such binders: (1) a de novo designed mini-binder proteins reported by Cao et al. and Linsky et al. that target RBD with IC50 values ranging from femtomolar to nanomolar; (2) several nanobodies that bind with nanomolar or better affinity; (3) short synthetic peptides that are highly tractable but tend to bind more weakly than proteins. Inventors highlight that one of the nanobodies inventors will investigate was trimerized using a Gly-Ser linker and achieved femtomolar binding affinity and picomolar virus inhibition, despite using a flexible linkage and linear concatenation via genetic fusion. Thus, our nanostructure-scaffolded, size/geometry-matched approach may give even greater affinity by reducing the entropic penalties for rearrangement to the correct geometry.

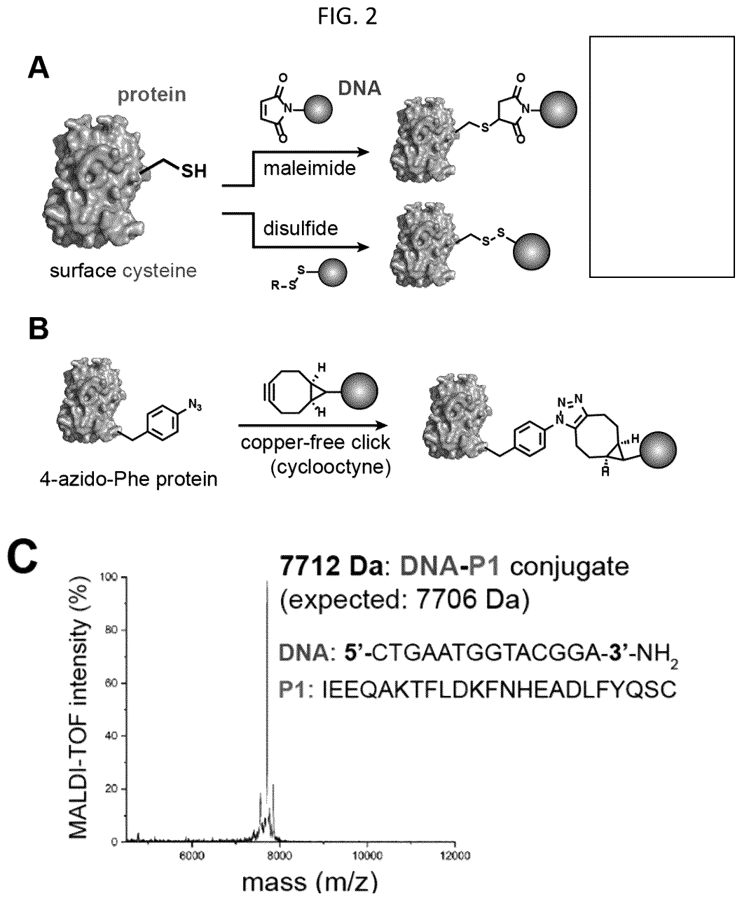

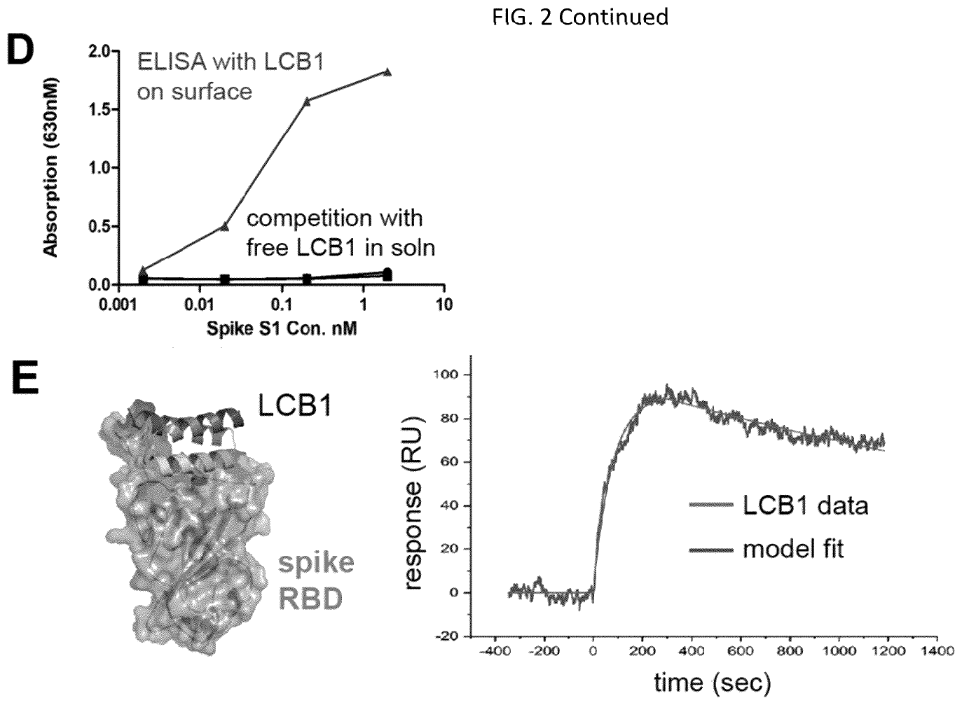

All polypeptides will be conjugated to DNA one of two ways, both of which have been extensively used in PI Stephanopoulos's lab: (1) via a unique, mutagenically-introduced cysteine using a bifunctional linker (FIG. 2A); and (2) copper-free click between a protein bearing the noncanonical amino acid 4-azidophenylalanine and cyclooctyne-DNA (FIG. 2B). Proteins will be expressed recombinantly in E. coli, and peptides will be synthesized on solid phase using standard Fmoc-protected amino acids. Conjugates will be purified using anion exchange or reverse phase chromatography, and characterized via polyacrylamide gel electrophoresis and MALDI-TOF mass spectrometry. The selected binding groups have a range of affinities (from picomolar to low micromolar), which will allow us to determine the range of affinity enhancements imparted by the multivalent scaffold. Recent experiments creating nanobody heterodimers using flexible amino acid linkers have shown affinity enhancements of 4-22 fold, so inventors expect constructs to be at least within this range, with potentially much higher affinities due to the better-defined 3D presentation of the ligands. Preliminary data: The LCB1 protein reported by Cao et al. via recombinant expression followed by nickel affinity and anion exchange chromatography methods has been successfully expressed. A unique mutagenic cysteine has been incorporated into this protein and coupled to DNA using a bifunctional linker. In addition, synthesis of synthetic peptides (up to 50 amino acids) with noncanonical azide residues for copper-free click coupling to DNA (FIG. 2C), as well as nanobody proteins for creating hybrid protein-DNA nanomaterials has been routinely performed.

Test RBD binding activity of peptides/proteins and DNA conjugates. To test the ability of the synthesized peptides/proteins to bind to the SARS-CoV-2 RBD, Inventors will employ two methods: (1) an ELISA assay using the RBD and its targeting antibody; and (2) surface plasmon resonance (SPR), which was used in the characterization of most of the binding groups mentioned above, and enables greater insight into on- and off-rates of the binding molecules. Preliminary data: Inventors have probed the binding of our in-house expressed LCB1 to RBD using both ELISA and an SPR assay. The LCB1 protein was adsorbed to the surface, followed by exposure to varying concentrations of the monomeric spike RBD protein; the amount of RBD adhered was then probed with a primary antibody and a secondary antibody-HRP conjugate. The RBD protein did indeed bind to the LCB1, with a Kd in the 100-200 pM range, similar to reported values (FIG. 2D, red curve). The binding could also be abolished by competition with free LCB1 in solution (FIG. 2D, black curve), further confirming that the RBD was not nonspecifically adsorbing to the surface. The binding was also be probed by SPR (FIG. 2E) and demonstrated a Kd˜9 nM, consistent with reported results.

Inventors will test the LCB1-DNA conjugate—and all the peptide/protein-DNA hybrids made in an analogous fashion—in the same manner, cognizant of the fact that the DNA handle could decrease the binding affinity. Although the attachment site for DNA has been engineered to be distant from the RBD-binding interface, it may be necessary to screen several attachment sites, as well as linker identities (e.g. alkyl, aryl, PEG) and lengths. Mutated peptide/protein molecules, where the binding interface residues are scrambled to abolish binding, will be used as controls. Inventors will use SPR to determine rates of binding (kon, koff), and thus the Kd values. All conjugates will be compared with the original (i.e. non-DNA-conjugated) binding groups as positive controls.

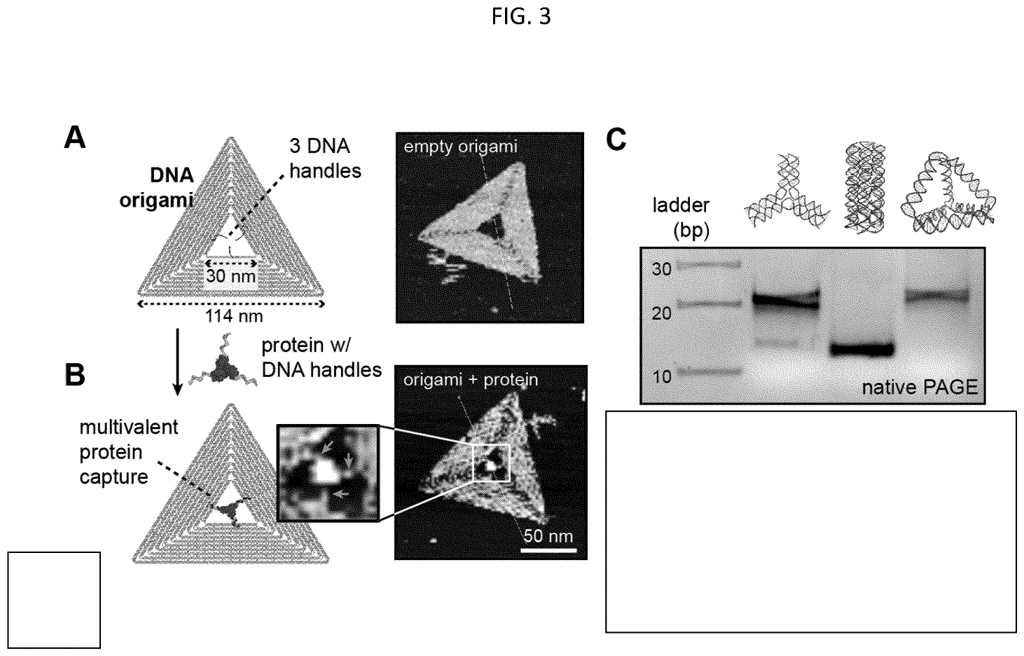

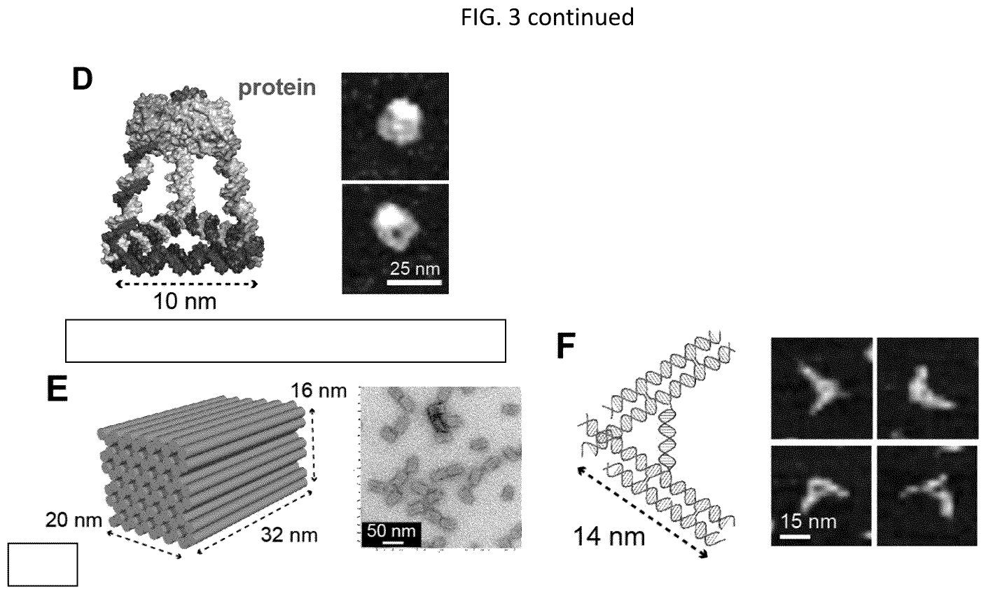

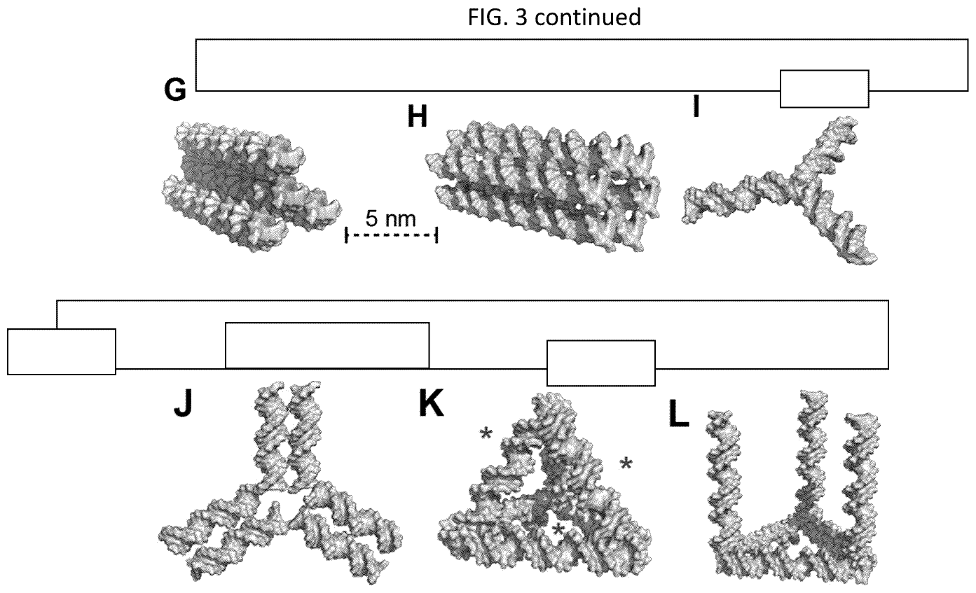

Design, synthesize, and characterize hetero-trivalent peptide/protein-DNA nanostructures. The ideal DNA nano-scaffold for hetero-trivalent presentation of the above peptide/protein-DNA conjugates is a structure that is reasonably rigid (to avoid entropic penalties in nanostructure reconfiguration), and roughly size-matched to the spike RBD trimer diameter (˜7-8 nm). Nanostructures scaffolds will be assembled using thermal annealing of the constituent strands, and purified using either spin filtration, gel excision, or anion exchange chromatography. Single-stranded DNA handles will be included for attachment of peptide/protein-DNA conjugates, and successful incorporation will be probed using gel shift assays and/or using fluorescently tagged peptides/proteins. Nanostructures with zero, one, and two handles for peptide/protein incorporation will be synthesized to probe the effect of not just binding group, but also valency; indeed, this straightforward tunability is an advantage of DNA nanoscaffolds. Preliminary data: A series of DNA nanostructures were designed and used, including (FIG. 3A-F): triangular DNA origami structures; tetrahedral wireframe cages, six-helix bundles, block-like origami cuboids, reconfigurable tweezers, double crossover tiles, and branched three-way junctions, among many others. These examples include both all-DNA nanostructures, as well as structures that precisely integrate proteins in a multivalent fashion (FIG. 3B,D) The lab has extensive experience with DNA design software (e.g. Cadnano, Tiamat) as well as techniques and access to facilities to analyze the nanostructures (gel electrophoresis, AFM, TEM).

For this work, inventors will primarily focus on simpler DNA nanostructures (rather than full-size origami) in order to better match the protein size, and to improve the overall scalability of the final assemblies. Towards this end, inventors will test structures like four- and six-helix bundles (FIG. 3G,H), which are cylindrical objects ˜5-7 nm in diameter and highly rigid due to multiple crossover strands linking them together. Inventors will also test three-way tile junctions, tetrahedral cages, and other wireframe assemblies (FIG. 3I-L) that vary in flexibility and shape. These shapes can be tuned over a range of sizes (5-20 nm, though larger structures are also possible with more complex designs), and the valency can be varied from 2-4 ligands readily. They each contain multiple sites for attachment of proteins/peptides; the simplest is at the end of helices by extending the structural strands with ssDNA handles, but the ligands can also be coupled directly to the structural strands and displayed at arbitrary locations along a helix (i.e. not just the ends) by introducing a nick point, or directly conjugated to the backbone of any constituent helix with single-nucleotide precision.

Develop a computational model for simulating hybrid peptide/protein-DNA nanostructures. One bottleneck to developing the proposed DNA-peptide hybrid molecules is that no model exists for the design of hybrid polypeptide-DNA nanostructures (unlike for proteins where packages like Rosetta50 exist for modeling structure and designing novel binding groups). Accurately representing the 3D spatial display of multiple heterogeneous molecules on a DNA nanostructure would allow more accurate matching of the hybrid structure to the target. Such a model would also enable the in silico “mutagenesis” and screening of designs that best match the binding sites on the target in order to guide experimental realization. Preliminary data: The oxDNA tool, a coarse-grained model of DNA that reproduces mechanical, structural and thermodynamic properties of both single-stranded (ss) and double-stranded (ds) DNA will be used. The model has been used in a range of settings, from biophysical studies of DNA to probing the assembly of nanostructures and active nanodevices, usually with good agreement with existing experimental data. OxDNA can efficiently simulate nanostructures consisting of up to tens of thousands of nucleotides and captures timescales that correspond to tens of milliseconds in experiment51. Recently, an extension of the model was introduced: ANM-oxDNA, that uses the oxDNA model for DNA and also represents protein structures and short peptides using the anisotropic-network-model (ANM) to capture their basic dynamics and conformations. The model is able to reproduce the structure of protein-DNA hybrid structures previously realized in Stephanopoulos lab. Currently, the model does not predict de novo interactions between peptides and proteins, and the possible interactions have to be explicitly specified based on prior knowledge of the binding sites. The model can, however, very quickly sample nanostructure diffusion well as its binding trajectory to a protein. Our prior analysis has shown that the simulation can efficiently sample the possible conformations of a DNA nanostructure—and the regions that a multivalent binder can cover on a protein—within less than 1 GPU-hour for a protein and a nanostructure system consisting of several hundred residues in total.

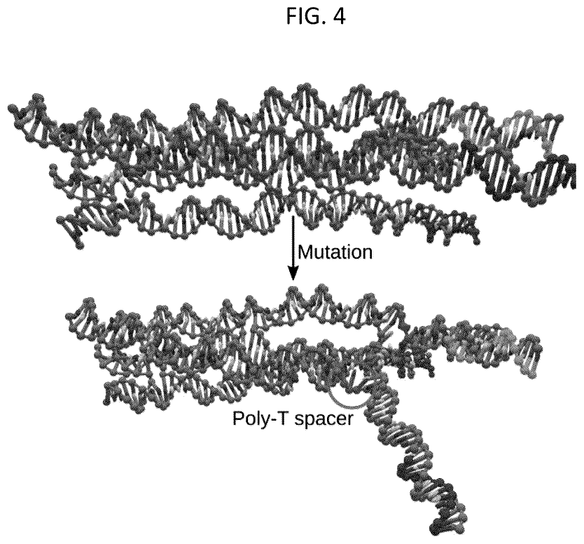

Here, inventors will implement an automated in-silico nanostructure mutation generation using our recently developed ox View design tool for nucleic acid nanotechnology, which was recently extended to also support protein structure representation. The initial design for a multivalent peptide/protein-DNA nanostructure can be either imported from other DNA nanotechnology design tools or created directly in ox View. Inventors will then implement an automated algorithm for introducing “mutations” to the structure design, which will include: changing the position for peptide/protein attachment, extending/shortening dsDNA and ssDNA segments in the nanostructure, and introducing bulges and junctions into the design (FIG. 4). Inventors will further implement a docking protocol that calculates the entropy difference between the bound and unbound structure, and enthalpy that is based on provided scoring function that canbe imported from peptide-protein docking tools.

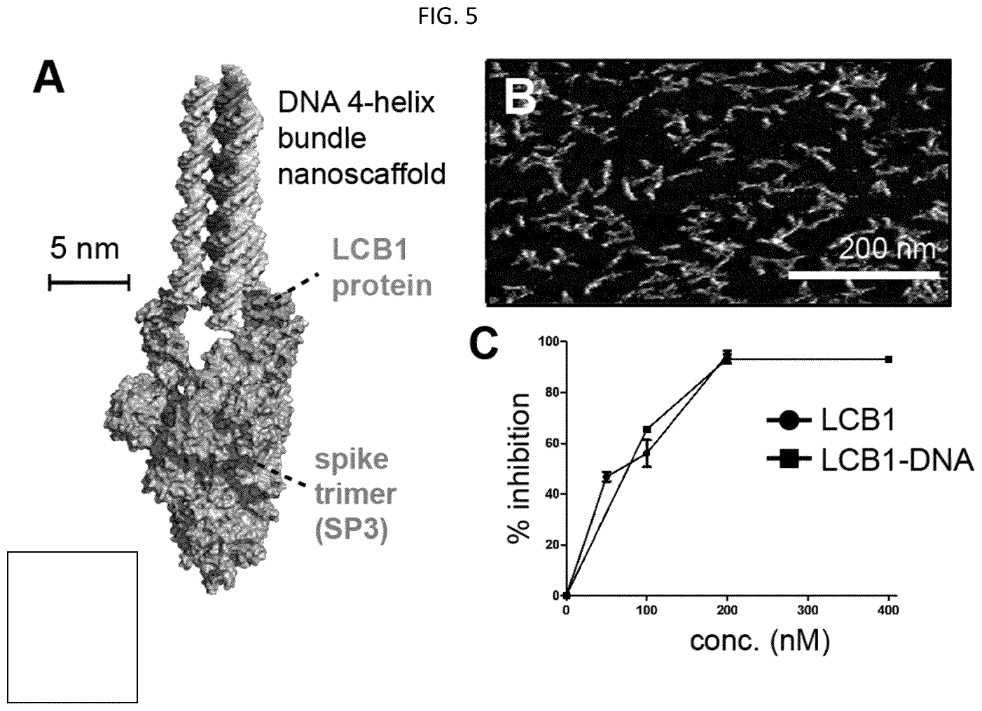

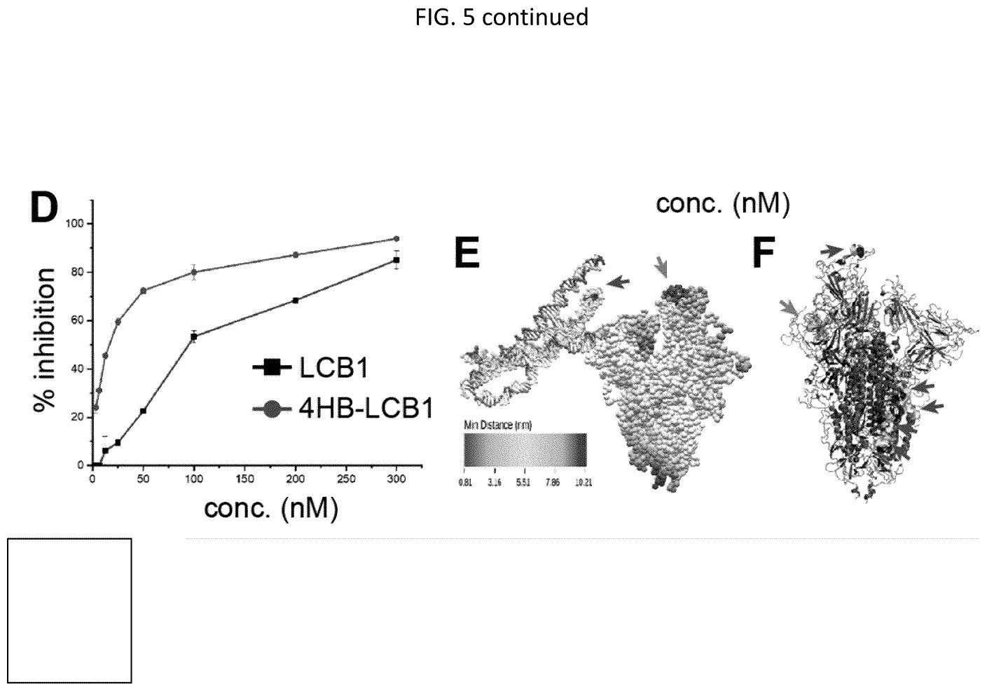

Use the experimental and computational pipeline to optimize DNA-peptide hybrid molecule structures. Following synthesis of hetero-trivalent DNA-peptide hybrid molecules bearing LCB1 or other RBD-binding domains, inventors will probe their binding to a homotrimeric spike protein complex (SP3), and use the computational model to guide nanostructure refinement and testing. This trimerized spike protein is available from commercial suppliers, and inventors will rationally design a set of starting designs, approximately positioning the binding peptides to match the position of the ACE2 binding sites on the SP3 (FIG. 5A). Inventors will then use the optimization platform to in silico “evolve” the strongest binder, where the scoring function will optimize both the entropy of binding (by minimizing the entropy loss when the DNA-peptide hybrid molecule is bound to SP3), as well as maximize binding enthalpy; e.g. if the structure is too rigid, the peptides will not be able to correctly dock into the binding site. Given the efficiency of the coarse-grained model, inventors will run thousands of rounds of DNA-peptide hybrid molecule in silico evolution to obtain the most promising candidate nanostructures (˜10-20 total) for experimental testing. For each designed structure to be probed experimentally, inventors will use the model to study its folding to make sure it is able to form correctly, and does not include alternative metastable misfolded states. Preliminary data: LCB1 has been conjugated to DNA handles, and incorporated it into three- and four-helix DNA bundles (FIG. 5A,B). The monomeric LCB1-DNA conjugate bound equally well as the protein alone (FIG. 5C). Inventors tested these bundles for binding to RBD via an inhibition ELISA assay, whereby RBD binding to immobilized LCB1 competed with soluble DNA-peptide hybrid molecules. Indeed, the trivalent DNA-peptide hybrid molecule bound RBD better than the monomeric LCB1 (FIG. 5D). Although these nanostructures are homo-trivalent, the spike RBD target is still monomeric; experiments are currently underway with the trimeric spike protein (SP3) to directly probe the size-matched binding and affinity enhancement of the DNA-peptide hybrid molecules.

A key feature of multivalent binding is not just enhanced affinity, but a greatly decreased koff for binding, e.g. as seen by Strauch et al. for homotrivalent HA binding proteins. Inventors will probe the binding kinetics of DNA-peptide hybrid molecules by SPR, and compare to nanostructures bearing only one or two peptides/proteins, and mutated (non-binding) molecules. While our model will not be able to directly predict the binding affinity, it will still be possible to rank the structures based on the scoring function. Inventors will compare the experimentally-measured binding affinity with the ranking produced by the model, and seek to adapt the scoring function to match the experiments. Thus, rather than creating a funnel-like approach commonly used in computational design pipelines—where a set of binders is generated, from which only subset is then successfully verified in experiment—our work will create a feedback design loop, where the efficient but coarse model is improved through experimental measurements. At the same time, the model will allow us to effectively search design space and provide iteratively improved designs for experimental probing. As well as determining the affinity between DNA-peptide hybrid molecules and SP3, inventors will also probe the structure's ability to block the spike trimer association with the ACE2 receptor, as a proxy for inhibiting viral infection. In addition to traditional binding/blocking studies via SPR, inventors will also probe the DNA-peptide hybrid molecule binding via negative stain transmission electron microscopy (TEM) and atomic force microscopy (AFM). Both DNA nanostructures and bound proteins can be readily visualized using these methods, so they can be used to demonstrate not just binding, but also affinity (e.g. by counting structures with and without proteins). Results will be compared to free proteins/peptides, and homo-trimerized binding groups using flexible chemical or genetically expressed linkers.

Determine distances between RBD site and a separate binding site. Most targets of interests are not homo-oligomeric, so the approach outlined above will not be applicable. Thus, inventors will develop a DNA-peptide hybrid molecule that can position two different targeting groups—where one has a known and the other an unknown binding site on the target—with 3D precision in order to enhance the affinity. Once again, inventors will use the SARS-CoV-2 spike protein as the target, because recently several nanobodies, a bispecific IgG mimetic, and an aptamer were reported that did not target the ACE2 binding domain on RBD. One nanobody in particular was shown to bind the N-terminal domain (NTD) of the spike protein, and did not compete with a separate nanobody that bound to the RBD. Inventors will use this NTD site as a test system to (1) develop new computational experimental method that will be able to de novo identify location of binding sites, and (2) create a DNA-peptide hybrid molecule that can position the two groups with spatial precision to match this experimentally-determined distance. Inventors will initially develop a computational-experimental pipeline to determine the location of the second binding site as if the NTD binding site was not known, allowing us to compare our unbiased results to the known location after the fact. The pipeline will generate a set of “nano-rulers,” consisting of the two binding groups linked by simple dsDNA linkers of known length. Inventors will annotate the possible binding sites using available peptides global docking tools that provide a list of approximately 4-10 candidate binding sites, featuring multiple false positives. Inventors will then use the computational platform to design a set of DNA scaffolds with the peptides attached at different distances. Thus, when one peptide (e.g. LCB1) is bound to the RBD, the second peptide on the scaffold covers different distances on the surface of the protein. The set of scaffolds will be designed to cover the respective possible binding distances between the known binding site and the candidate binding site. By comparing the experimental affinity measurements between the designed scaffolds, inventors will be able to select the scaffold that binds to both sites at the same time, and thus “identify” (i.e. confirm) the position on the second binding site (FIG. 5E,F).

Design and test hetero-bivalent DNA-peptide hybrid molecules to match the distances determined. Once the approximate distances and location have been determined for the two binding groups, inventors will design heterobivalent DNA-peptide hybrid molecules that recapitulate this distance and probe for both binding and blocking of the structure to the spike RBD monomer. Although the nanostructures discussed previously can be used, inventors will also explore simpler structure like rigid double-crossover (DX) tiles, where the binding groups can be positioned at multiple locations. The tile will provide added steric bulk for blocking the interaction with ACE2. Inventors will follow the same computational-experimental pipeline as presented previously, and compare the DNA-peptide hybrid molecules to binding groups dimerize using flexible linkers (either alkyl, PEG, or amino acid (via recombinant expression)).

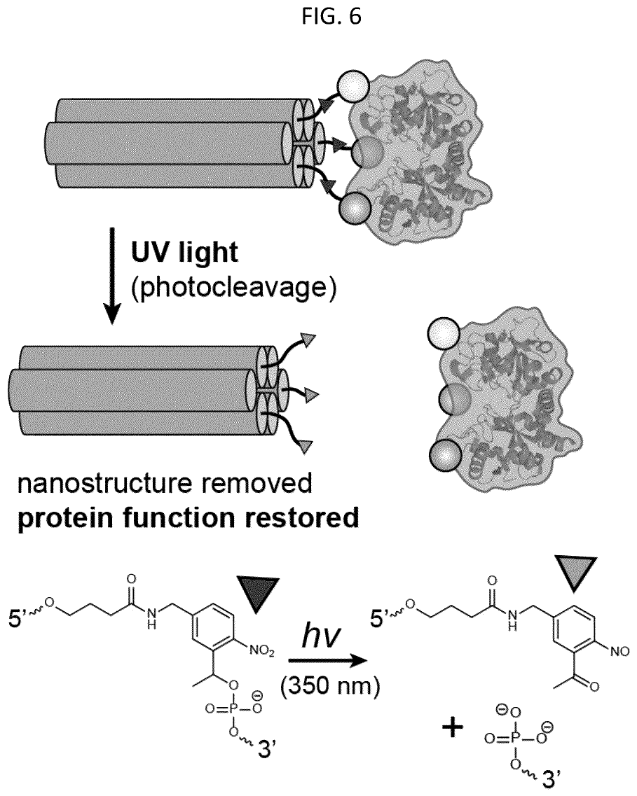

Demonstrate stimulus-responsive “off” switch for DNA-peptide hybrid molecule binding. One key advantage of our approach for blocking protein function is that it can, in principle, be reversed by disassembling the nanostructure in a stimulus-responsive fashion. In particular, o-nitrobenzyl ester moieties can be installed into the DNA backbone, resulting in clean scission upon exposure to 350 nm UV light, an approached used by PI Stephanopoulos to install photocleavable functionality into a DNA nanomechanical device. Inventors will incorporate such a photocleavable moiety into the DNA handles attached to the binding groups from presented earlier, so upon UV illumination the entire scaffolding DNA nanostructure is released (FIG. 6). This approach leaves behind the bound peptides/proteins, so it may be necessary to use the lower-affinity binding groups, as opposed to the already high-affinity proteins like LCB1. Earlier, where the binding groups may not target the key protein interface, such a photocleavage will still expose that interface by simply removing the steric blockage imparted by the nanostructure. Inventors will use SPR to probe this UV triggered “activation,” immobilizing the ACE2 receptor on the surface and adding a solution of SP3 pre-blocked using the trivalent DNA-peptide hybrid molecule. Upon UV illumination, the nanostructure should be removed (with a timescale of several seconds for photocleavage, according to our previous work) and the kinetics of ACE2-SP3 binding will be monitored by SPR.

Expected outcomes, potential pitfalls, and alternative approaches: By the end of this work, inventors will have developed DNA nanostructures bearing: (1) three copies of an RBD-2 binding peptide/protein, or (2) two different binding groups for the spike protein. Inventors will have optimized the computational pipeline to in silico evolve these nanostructures by comparing their bound and unbound state, which is efficient enough to run freely diffusing simulations of the binding trajectory. Potential pitfalls and alternate solutions include the following. (1) Attachment of a DNA handle may compromise peptide binding. While inventors expect that folded proteins like LCB1 will not be greatly affected by DNA handle attachment, it is possible that shorter peptides may be more sensitive helix stabilizing residues or backbone (i, i+7) crosslinks, or use neutral peptide nucleic acid (PNA) handles instead of anionic DNA. (2) The simulations might incorrectly predict the affinity of the designed nanostructures. In that case, inventors will use the experimentally measured affinity to further update the scoring function that will be used in the simulation to assess the enthalpic contribution of binding to the protein surface. (3) The proteins used are too large to effectively position them in 3D space. It is possible that, especially for targeting two different spots on the spike monomer, using LCB1 and a nanobody (or two nanobodies) will be too sterically bulky. In this case, inventors will use cyclic peptides recapitulating the CDR3 loop from the nanobodies, as described in greater length previously. (4) The SARS-CoV-2 spike protein is a poor target. If no successful hetero-bivalent DNA-peptide hybrid molecules against the spike protein are found—e.g. because distances determined previously are too small for a DNA nanostructure to effectively bind—inventors will instead turn to a different target: influenza hemagglutinin (HA). Indeed, trivalent protein scaffolds with grafted CDR loops have demonstrated high-affinity binding to this target3, so inventors will use the same loops as starting points for our design. Furthermore, a number of short peptides discovered from on-chip peptide arrays have been reported for HA. Inventors will carry out our “molecular ruler” method for these peptides to find combinations that span distances suitable to DNA nanostructures. Most of these peptides have only modest affinities (Kd˜low micromolar), so attachment to a scaffold could increase the affinity to/past the nanomolar regime, as demonstrated using chemical linkers.

Develop a photo-switchable blocking DNA-peptide hybrid molecule for fibrinogen. Rationale: If multiple binding agents are not readily available for a target, one or more must be discovered using selection methods like phage display. However, this approach poses the challenge that the binding sites for these new targeting groups are not known, and thus must be determined prior to incorporation into a scaffolding nanostructure (which will itself be tuned to best recapitulate these distances). Inventors will work to discover new binding peptides for fibrinogen, in order to block its assembly into fibrin clots. These peptides will not all bind in the same location, so the methods developed in previously will be employed to map their likely distances on the target, in order to design a heterobi- or tri-valent DNA-peptide hybrid molecule that can inactivate the protein-protein interactions. Inventors will also use the photocleavable approach described previously to “turn on” fibrin self-assembly by unblocking the structure. Although Inventors will focus on fibrinogen as a proof of principle, Inventors will have developed a pipeline for future targeting of any protein through a three-step process: (1) Identify a subset of binding nanobodies/peptides against the target; (2) Determine the pairwise distances for proteins/peptides that bind to nonoverlapping sites; and (3) Design a DNA-peptide hybrid molecule to effectively envelop the target, using the computational experimental approach outlined earlier.

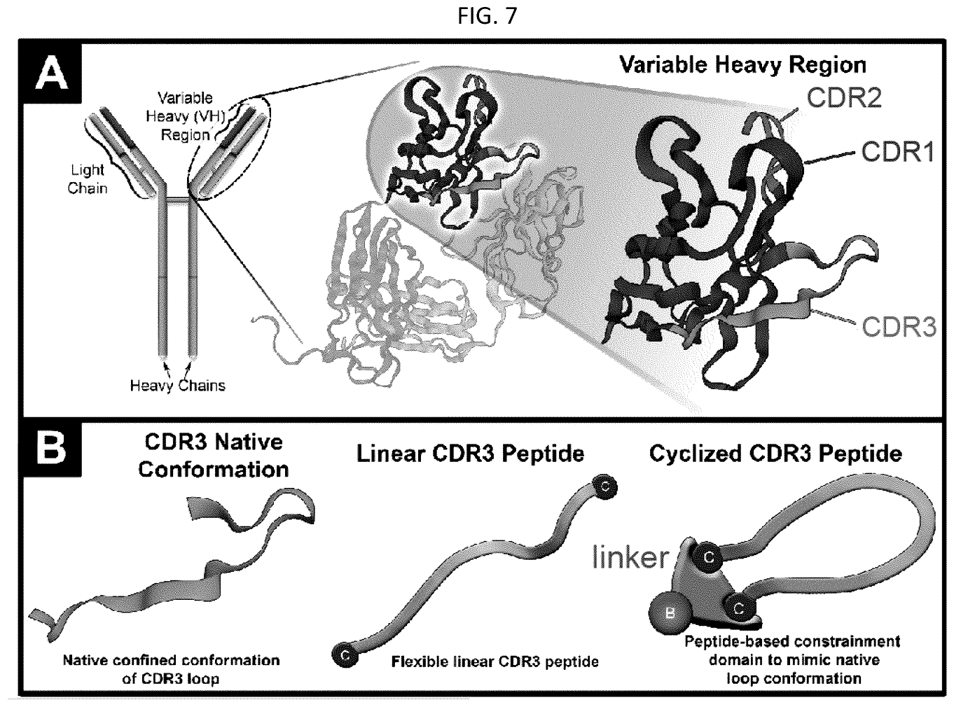

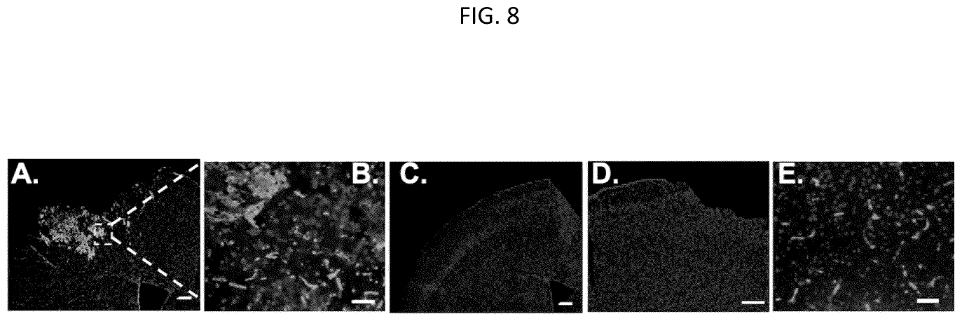

Preliminary data: Phage display can be used to find novel targeting nanobodies against complex targets such as fibrin, in vitro cell culture models of reactive astrocytes, ex vivo tissue sections from small and large animal models of brain injury, and in vivo brain injury mouse models. However, the target nanobodies are often difficult to express recombinantly, leading to poor yields or aggregation. Thus, it was recently reported that cyclized peptides from the CDR3 loop of targeting nanobodies can be highly effective as targeting agents, while retaining a small size and ease of synthesis. This approach was termed the CDR3 Loop Assembly via Structured Peptide (“CLASP”) system (FIG. 7). Inventors demonstrated such power of phage display by identifying CDR3 motifs with a domain antibody phage library (dAb) that recognize temporal alterations in the neural injury microenvironment (FIG. 8). Inventors conducted three in vivo phage biopanning screens with the dAb phage library in mice that sustained a focal TBI (controlled cortical impact; CCI) at three different time points post-injury (1, 7, and 21 days post-injury; dpi). Using next generation sequencing and bioinformatics analysis, Inventors then compared and identified enriched phage populations for each time point post-injury (FIG. 8). The bioinformatic analysis focused on ranking by CDR3 as this region imparts high diversity and specificity for dAb/antigen recognition compared to CDR1 and CDR2. This analysis pipeline enabled selection of prominent CDR3 targeting domains for either acute injury (1 dpi) and subacute (7 dpi). The discovery was further made possible by applying strict selection criteria to identify top candidate CDR3 sequences for further characterization for each time point. The selection criteria included: (1) unique to a distinct temporal phase post-injury, (2) not present in control phage libraries (amplified without biopanning), or peripheral tissue (heart, liver, spleen), or sham library, and (3) high frequency and enrichment observed round to round. After applying this selection criteria, Inventors used the CLASP system to generate CDR3 mimetics for validation testing (FIG. 7B). Ultimately, Inventors successfully identified and validated two CLASP cyclic peptides that recognize acute (1 dpi) or subacute (7 dpi) TBI. The immunohistochemical based assessment on post-mortem murine TBI tissue presented in FIG. 8 demonstrate the stark temporal and spatial localization to neural injury by the acute and subacute CLASP motifs. By using synthetic peptides, it will also be possible to explore nanostructure design and tighter integration of the peptides into the DNA scaffold to better mimic loop placement on antibodies. Here, in this application, Inventors will leverage extensive experience with fibrin/fibrinogen targeting and polymerization dynamics to focus on fibrin as a proof of principle to develop a pipeline for future targeting of any protein of interest.

Phage display against key fibrinogen polymerization domains to discover nanobody CDR3 loops. Inventors will leverage prior knowledge of the fibrin knob-pocket interactions that drive fibrin assembly and polymerization; specifically, Inventors will use the short peptide sequence of GPRPXX (SEQ ID NO: 3) that recognizes hydrophobic pocket domains on the beta and gamma chains. Phage display with the aforementioned dAb phage library against fibrinogen in the presence of the GPRPXX (SEQ ID NO: 3) peptide (at millimolar concentrations to compensate for its modest Kd (5-10 μM) will be conducted to identify recognition domains outside of the pocket regions. Human fibrinogen will be immobilized on microbeads via EDC/NHS chemistry. Inventors will carry out biopanning with a naïve human dAb phage library, which will be produced and purified per protocol. Substrates will be incubated with dAb phage (100 μl of 1010-1012 CFU) for 1 hr. Non-specific binding phage will be removed via a series of rinses with PBS+0.1% Tween 20 (PBST). The target bound phage will then be eluted, collected, and amplified. Subsequent rounds will be repeated with an enriched population of eluted phage from the previous round. A minimum of three biopanning rounds will be completed, with a goal of obtaining 10-20 nanobodies that span a range of binding areas on the protein. To identify the CDR3 loop, Inventors will carry out next generation sequencing (NGS) and bioinformatic analysis. The use of NGS provides a robust and high-throughput alternative to Sanger sequencing with extensive coverage, enabling an in-depth analysis on the eluted phage libraries. Here, amplified plasmid DNA from the eluted phage libraries will be prepared for Illumina MiSeq 2×250 sequencing. Paired end sequences will be stitched together using Fast Length Adjustment of SHort Reads (FLASH). HCDR3 sequences will be clustered using a hierarchical Levenshtein Distance algorithm with FASTApatmer Perl scripts. Each library will be searched for HCDR3 sequences that are enriched through the biopanning round using a combination of in-house R scripts and Galaxy modules. The top enriched dAb sequences will be selected based on the HCDR3 analysis and the following selection criteria: 1) unique to a distinct target, 2) not present in control phage library (amplified without biopanning), and 3) high frequency and enrichment observed round to round.

Synthesis of cyclic peptides and DNA conjugates. Following identification of nanobody-derived CDR3 loops that bind to fibrinogen, Inventors will next synthesize cyclic version of these peptides by introducing terminal cysteine residues and bis-bromoacetamide linkers as described previously. The linkers will also incorporate linear alkynes for copper-catalyzed click coupling to azide DNA. Peptide-DNA conjugates will be purified and characterized as described in earlier, and individually tested for binding to fibrinogen by SPR (both cyclic peptides alone and DNA conjugates thereof).

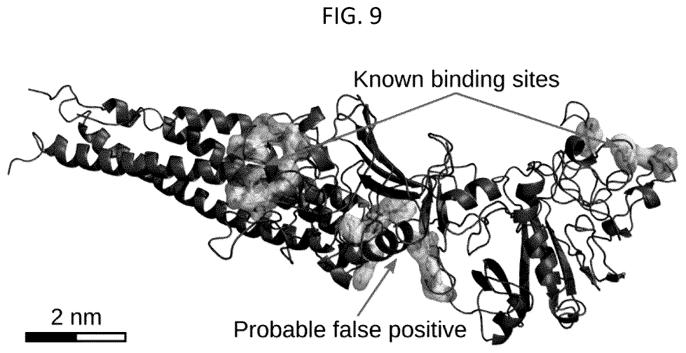

Determination of pairwise distances for three-peptide sets. Inventors will next use the set of peptide-DNA conjugates to map out potential binding sites to fibrinogen, as outlined above. One of the peptides used will be the GPRPXX (SEQ ID NO: 3) sequence that binds to the pocket domains, and it will serve as a way to “pin” the possible distances covered by the other peptides. Following the experimental-simulation pipeline developed in a first aspect, Inventors will use available global docking tools to annotate likely binding sites for the peptides identified to bind to fibrinogen in the phage display experiments (FIG. 9), and will develop a set of DNA “nanorulers”, where one end functionalized with GRPRXX (SEQ ID NO: 3) peptide binds to the pocket domain, and the other ends are designed to bind to one of the candidate binding sites. Using in silico evolution, Inventors will develop the rulers to only bind to the known and candidate sites, and test the set of nano-rulers in experimental measurement in affinity, identifying the ones with the highest affinity that correspond to the nano-ruler binding the GPRPXX (SEQ ID NO: 3) binding site and the candidate site on fibrinogen.

Design and testing of hetero-trivalent DNA-peptide hybrid molecules. Inventors will use in silico iterative evolution framework developed in earlier to design candidate DNA nanostructures that position the peptides in 3D space. These structures will constrain GPRPXX (SEQ ID NO: 3) and, ideally, two additional CLASP peptides at the distances determined previously, and binding to fibrinogen will be probed using SPR as described in a first aspect. In addition, Inventors will probe the functional blocking of fibrin polymerization by the nanobody, following proteolytic cleavage by thrombin, using a suite of fibrin polymerization assays. Specifically, Inventors will assess polymerization dynamics (turbidity and thrombin clotting time), extent of clottable protein, and clot structure (confocal microscopy). Fibrin polymerization assay: Thrombin-initiated fibrin polymerization assays will be used to evaluate anticoagulant activity. For all assays, fibrin clots will be prepared with final concentrations of human fibrinogen at 1 mg/mL (plasminogen-, fibronectin-, von Willebrand Factor-depleted), human α-thrombin at 1 NIH U/mL (ERL), activated human factor XIII at 1 U/mL in a HEPES-buffered solution supplemented with calcium chloride. Prior to initiating polymerization, 50 μL of fibrinogen or fibrinogen+hetero-trivalent DNA-peptide hybrid molecules will be incubated at room temperature for 30 min in a transparent 96-well plate. Polymerization will be initiated by adding 50 μL of thrombin+FXIIIa to each well. Turbidity curves will be generated from absorbance measurements recorded every minute for 60 min at 350 nm. Post-assay analysis of turbidity curves will include the peak absorbance and thrombin clotting time. Percent clottable protein: Upon completing turbidity assays, the resulting fibrin clots will be removed, leaving behind the remaining soluble protein (i.e., the clot liquor). The soluble protein content in the clot liquor will be quantified using a Quant-iT protein assay (Invitrogen). Data will be assessed as percent clottable protein, the amount of initial protein minus soluble protein in the clot liquor all divided by the initial protein. Fibrin fiber structure: Confocal microscopy will be used to evaluate the fibrin fiber structure. Briefly, fibrin clots will be prepared as described above with addition of 5% fluorescently labeled fibrinogen. Upon initiating polymerization with thrombin and FXIIIa, 100 μL will be immediately transferred to a glass slide with 300 μm spacers and capped with a cover slide. Clots will be imaged 60 min after polymerization. Five random 10 μm zstack sections of each clot will be imaged with a Zeiss Laser Scanning Microscope. Image analysis and 3D projections will be performed with ZEN imaging software.

Reversible blocking of fibrin assembly. As outlined above, an advantage of our approach is the ability to cleave the nanostructure for the binding peptides/proteins using UV light. Given that some of the peptides discovered herein will bind to sites away from the key binding interface, it is likely that photo-removal of the DNA scaffold will restore binding even if the individual peptides remain bound. In particular, the weak (micromolar) affinity of GPRPXX (SEQ ID NO: 3) for the pocket suggests that upon nanostructure cleavage, this peptide will dissociate from the protein without the avidity effects of the other binding groups. Pre-blocked fibrin will be cleaved using thrombin as above, and then exposed to UV light to remove the DNA-peptide hybrid molecule. The kinetics of polymerization will be compared with unblocked controls, and the fibrin fibers examined.

Expected outcomes, potential pitfalls, and alternative approaches: Inventors will have developed a novel DNA-peptide hybrid molecule that positions up to 3 cyclic peptides derived from phage display to block fibrin assembly until activated using light. Potential pitfalls and alternate solutions include the following. (1) Phage display against the fully intact fibrinogen protein does not yield relevant CDR3 domains. If the CDR3 domains cannot block polymerization, Inventors will use enzymatically or chemically cleaved fragments of fibrinogen to further refine/constrain the target to the pocket domain (i.e. fragment D). (2) Global docking tools are unable to give sufficient number of candidate binding sites and none of the designed nano-rulers can successfully identify the binding sites of the CD3 loops. In such case, Inventors will design in silico a set multivalent nanostructure functionalized with CDR3 loops selected against chemically cleaved individual fragments of fibrinogen. Inventors will optimize the nanostructure so that its respective arms with attached CD3 loop are designed to cover the entire protein fragment against which the CDR3 loop was selected. (3) DNA conjugation perturbs cyclic peptide binding affinity. If the DNA handles reduce or abolish the CLASP peptide binding, Inventors will explore constructs with varied linker lengths, or use PNA handles instead of DNA to avoid charge repulsion. It may also be necessary to append both ends of the peptide directly to the DNA backbone (using the structure to effectively cyclize it) in order to reduce flexibility in the system.

The two broad aspects of the disclosed technology outlined above rely on knowing individual binding proteins or peptides. A more powerful method, however, would be to directly select the bi- or tri-valent nanostructure, by creating a combinatorial library of all possible peptide/protein combinations on the scaffold. Inventors envision creating libraries of formed nanostructures with random combinations of peptides/proteins and selecting for the final assembly. For instance, peptide-RNA conjugates generated from mRNA display can be integrated into the scaffolds through a common poly(A) linker. However, the nanostructures used to scaffold these peptides/proteins will be tunable from the outset to match the rough size of the target, and a subsequent optimization of the scaffold could be performed to further enhance binding. Following selection of the best heterotrivalent nanostructure, the peptide identity can be deduced via sequencing of the appended mRNA handles. Finally, our approach can be used to block previously un-targetable proteins; by using any surface on the protein as a “handle” to help associate a nanostructure and block a key interface, Inventors expand the space of targetable protein patches. The use of multiple binding sites to enhance affinity can also reduce mutational escape if any patch changes, and allow the combination of peptides, aptamers, and even small molecules on the scaffold.

### Example 2

### High Sensitivity DNA Linked Immunosorbent Signal Amplification Assay (DLISA) for Detection of Infectious SARS-Cov-2 Virus and Variants

Diagnostic tests for SARS-CoV-2 infection belong to three categories: (1) nucleic acid amplification tests, which detect the presence of virus RNA by reverse transcription-polymerase chain reaction (RT-PCR); (2) tests detecting the presence of viral antigens; and (3) tests detecting the presence of serological antibodies against SARS-Cov-2 antigens. The WHO recommends nucleic acid detection of SARS-CoV-2 in respiratory samples for the diagnosis of the virus. However, there remains a great need for serological assays that measure viral antigens, variants, and that can determine infectious seroconversion. Because the serological assays allow us to assess the seroconverts and transmission of the virus and its variants, serosurveys will allow us to determine the actual rate of infection and accurate infection fatality.

Serological assays for SARS-CoV-2 are becoming available, including ELISA, lateral flow assays, and virus neutralization assays. SARS-CoV-2 antigen-detecting rapid diagnostic tests (Ag-RDTs) provide potent tools for pathogen detection at the point of care, and facilitate public health intervention. Nonetheless, the majority of Ag-RDT validation studies were done before the emergence and subsequent dominance of SARS-CoV-2 variants of concern (VOC). For these variants, there is evidence of increases in transmissibility, more severe disease (e.g., increased hospitalizations or deaths), a significant reduction in neutralization by antibodies generated during previous infection or vaccination, reduced effectiveness of treatments or vaccines, and failure of diagnostic detection (https://www.cdc.gov/coronavirus/2019-ncov/variants/). Currently, there are few regular diagnostics for SARS-CoV-2 variants of concern (VOCs). A recent study compared seven commercially available SARS-CoV-2 antigen diagnostic tests against an established RT-PCR assay. The reports showed that the sensitivity range of most antigen tests overlaps with SARS-CoV-2 viral loads observed in the early week of symptoms in the infectious period in most patients. All of the Ag-RDT is based on the antibody-depended assay. Recently, the Baker lab demonstrated that there is a de novo designed mini-binder termed LCB1, a small and hyper-stable protein that targets the spike protein with high-affinity (K_{D}˜1 nM)¹². The Manglik lab developed nanobodies that bind to a different epitope of the spike protein by screening a yeast surface-displayed library of synthetic nanobody sequences. Both the mini-binder and nanobodies have higher affinities than the commercial antibodies against the spike protein. Our preliminary data show that the mini-binder has high-affinity binding with spike protein and variants (K_{D}˜400 pM). It is possible that Inventors can develop a high-sensitivity and low-cost sandwich serological assay for SARS-CoV-2, based on mini-binder and nanobodies as replacement antibodies.

DNA has emerged as an exceptional molecular building block for amplifying fluorophore molecular assemblies, via branched DNA assemblies¹⁴, the hybridization chain reaction (HCR)¹⁵, and immunostaining with signal amplification by exchange reaction (Immuno-SABER)¹⁶, which triggers the assembly of multiple fluorophores. Inventors have designed a High Sensitivity DNA-linked Immunosorbent Signal Amplification Assay (DLISA) based on a high-affinity spike protein mini-binder, nanobody, and controllable DNA-fluorophore signal amplification method, which is free of enzymatic reaction.

As shown in FIG. 1, the sandwich DLISA assay, including nanobody coating on a plate as a capture domain, mini-binder (or trivalent PDbody as outlined in a separate disclosure) conjugated with DNA-fluorophore as an amplified signal probe. Inventors choose the nanobody as a capture domain because it possesses high-affinity binding with N-term binding domain (NTD) of spike protein, whereas the high-affinity LCB1 mini-binder targets the RBD domain of the spike. The LCB1 mini-binder can be conjugated with ssDNA, or attached to a trivalent nanostructure, and annealing with DNA-fluorophore as an amplified signal probe will enhance the sensitivity and specificity in the identification of SARS-CoV-2 variants of concern (VOC). In brief, the DLISA assay has three significant advantages over existing SARS-CoV-2 antigen-detecting rapid diagnostic tests: 1) The DLISA assay can be highly effective in the serosurvey of infectious SARS-CoV-2 variants. The high affinity of LBC1 binds with variants that result in increased infectivity and transmissibility; 2) the assay can be highly sensitive and specific, based on high-affinity spike RBD mini-binder; 3) This assay significantly decreases cost compared with antibodies, as both LCB1 and the nanobody are obtained by recombinant expression in E. coli.

Design and development of DNA-linked Immunosorbent Signal Amplification Assay (DLISA). The sandwich DLISA assay constructs the captured nanobody and mini-binder conjugated with a DNA-fluorophore probe. 1) Nanobody coating on plate wells as a capture domain, high-affinity binding with the spike protein's N-term binding domain (NTD). 2) High-affinity spike RBD mini-binder, conjugated with DNA annealing with DNA-fluorophore as an amplified signal probe. Our preliminary data show that the mini-binder has a robust binding affinity down to the picomolar regime for spike and variants. To overcome the multiple spike variants escaping the neutralization antibody, significantly reducing the effectiveness of treatments and diagnostic detection failure, Inventors hypothesize that our DLISA assay could provide highly sensitive, specific, and efficient targeting of SARS-CoV-2 variants of concern for the serosurvey against a SARS-Cov-2 virus variant infection.

Validation and characterization of mini-binder DNA-fluorophore signal amplification by primer exchange reaction (PER). The mini-binder DNA probe signal amplification relies on controlled in vitro synthesis of amplifier concatemers by PER. PER utilizes a catalytic hairpin template for controlled extension of a shorter primer sequence in a repeated manner. Our preliminary data show: 1) The LCB1 mini-binder has a robust binding affinity up to picomolar for spike and spike variants; 2) Mini-binders have been very highly efficiently conjugated with DNA sequences via crosslinkers; 3) The long DNA concatemers have been obtained by a simple one-step synthesis, but the desired lengths reaching >500 nucleotides may need to be optimized by external parameters, such as dNTP, hairpin design, reaction time, and temperature. 4) For application on biological specimens, the DLISA assay testing specificity, resolution, and amplification efficiency will be developed and optimized.

Development of Fast-simple DLISA assay. Based on DLISA assay, Inventors will develop a series of fast-simple DLISA assays, such as a microfluidic digital DLISA and a smartphone DLISA via small filter lens device assay in FIG. 2. Inventors will also assess the fast-simple DLISA assay testing specificity, sensitivity and resolution efficiency.

Validation of Fast-simple DLISA assay for clinical samples. Based on fast-simple DLISA assay, Inventors will assess the fast-simple DLISA assay testing specificity, sensitivity and resolution efficiency in a clinical SARS-CoV-2 sample, including serum, saliva, and nasal swab samples.

### Example 3

Many assays for SARS-CoV-2 testing face challenges for detection of variants. Nucleic acid (NAT), antibody response, and antigen rapid diagnostic tests (Ag-RDT) are widely applied to diagnose coronavirus disease 2019 (CovID-19). Currently, the nucleic acid test is the clinical gold standard for SARS-CoV-2 detection. Nucleic acid tests primarily detect the SARS-CoV-2 RNA genome, whereas antibody response and Ag-RDT tests detect the glycoprotein termed spike protein (S, a homotrimer), which mediates binding to host cells via the receptor angiotensin-converting enzyme 2 (ACE2). Limitations of Ag-RDT include: 1) SARS-CoV-2 viruses mutate with high frequency, yielding variants like Covid-19 alpha (B1.1.7) from the UK, Covid-19 beta (B1.351) from South Africa, Covid-19 gamma (P1) from Brazil, and Covid-19 delta (B1.617.2) from India, among others. 2) These mutations in the spike protein raise concerns that they will not be targeted by neutralizing monoclonal and vaccine-induced antibodies. Emerging SARS-CoV-2 variants can be problematic as they can result in changes that make the virus more likely to evade diagnostic tests. 3) Antibody tests do not detect the presence of the SARS-CoV-2 virus to diagnose COVID-19. These tests can return a negative result even in infected patients, if antibodies have not yet developed, or they may generate false positives. There is a great need for high sensitivity antigen assays, which measure virus and variants, to determine infectious seroconversion.

High-sensitivity antigen detection is critical for patient point-of-care testing and early diagnosis of SARS-Cov-2 disease, along with presymptomatic and asymptomatic identification for monitoring, forecasting, and ideally limiting epidemics. Serological antigen assays for SARS-CoV-2 are becoming available, including ELISA, lateral flow assays, and virus neutralization assays. SARS-CoV-2 antigen-detecting rapid diagnostic tests (Ag-RDTs) provide potent tools for pathogen detection, including at the point of care, and their use facilitates public health interventions. However, the majority of Ag-RDTs are qualitative and semi-quantitative assays, for which validation studies were performed before the emergence of SARS-CoV-2 variants of concern (VOCs). These VOCs have shown evidence of increased transmissibility, more severe disease (e.g., increased hospitalizations or deaths), and a significant reduction in diagnostic detection (https colon//www dot cdc dot gov/coronavirus/2019-ncov/variants/). Currently, there are few diagnostics for SARS-CoV-2 variants of concern (VOCs). A recent study compared seven commercially available SARS-CoV-2 antigen diagnostic tests against an established RT-PCR assay. The reports showed that the sensitivities of most Ag-RDT were less than 80%. These approaches rely on the affinity of the antibodies, which could limit test performance of Ag-RDT due to the emergence of VOCs that might not be effectively targeted by existing diagnostic antibodies.

Inventors disclose the use of a de novo designed mini-binder protein and nanobodies, in lieu of antibodies, to develop a novel assay for SARS-CoV-2 VOCs. The mini-binder targets the spike protein receptor binding domain (RBD) with high affinity (KD˜1 nM), and is also smaller and more stable than antibodies. The nanobodies bind to different epitopes of the spike protein—including ones separate from the RBD—as determined by screening a yeast surface-displayed library of synthetic nanobody sequences. Both the mini-binder and the nanobodies have a higher affinity than the commercial antibodies against the spike protein. Our preliminary data show that the mini-binder has high-affinity (K_{D}˜400 pM) binding with both the wild-type spike protein and several variants.

Based on a method termed primer exchange reaction (PER), one promising detection strategy has been tissue immunostaining with Signal Amplification By Exchange Reaction (Immuno-SABER), which is capable of sensing and highly multiplexed signal amplification in the local tissue environment. Inventors focused efforts on devising a linear, low-cost, quantitative assay to improve the sensitivity of the SARS-CoV-2 spike protein antigen test, ideally in a format suitable for both laboratory and rapid patient point-of-care applications. Disclosed herein is a High Sensitivity DNA-linked Immunosorbent Signal Amplification Assay (DLISA), based on a high-affinity spike protein mini-binder, nanobody, and controllable DNA-fluorophore signal amplification method used in Immuno-SABER, which is free of antibodies and enzymatic reactions.

Imaging-based digital immunoassay for rapid and sensitive biomarker detection. Detection and quantification of molecular biomarkers is critical to disease diagnosis and progression monitoring. Among the various developed approaches, ELISA (enzyme-linked immunosorbent assay) is the most well-established technology, amplifying antibody-biomarker binding via enzymatic reactions and converting reaction products into an optical signal (e.g., color changes). Although ELISA is widely used in clinical and research labs, its detection limit, total test time, and dynamic range are often insufficient for clinical applications. Recently developed digital immunoassays have improved the detection limit by measuring the binding of a single biomarker molecule to a capture antibody. Imaging-based digital immunoassays for rapid, sensitive, and precise detection of blood biomarkers with high clinical values have been developed. The method uses gold nanoparticle-labelled detection antibodies and optical imaging-based digital counting for real-time quantification of protein biomarker concentrations in as little as 1 μL of blood sample, and can eliminate non-specific binding signals via gradient based differential detection. Inventors disclose that integrating this digital immunoassay method with DLISA to create a point-of-care solution for rapid SARS-CoV-2 antigen and virus detection assays that works for all VOCs.

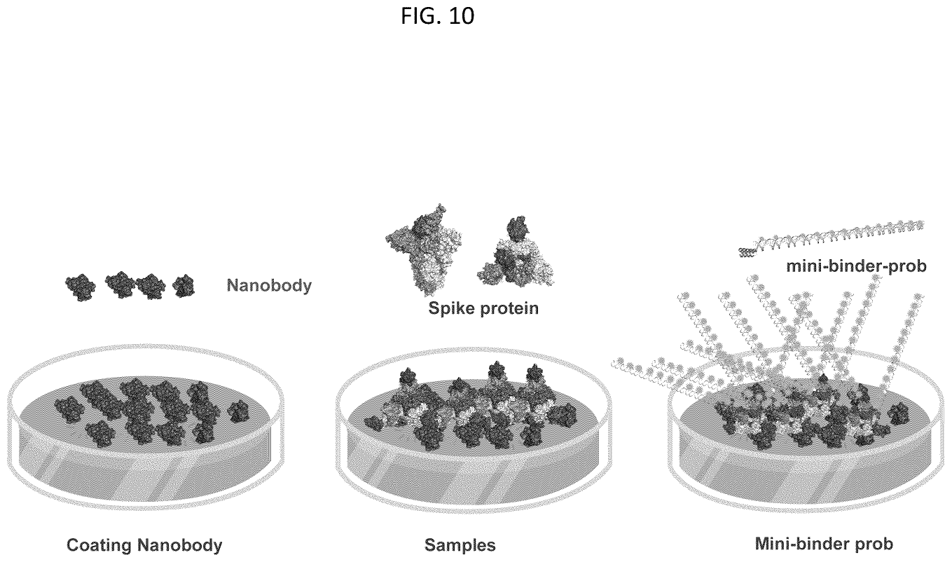

Herein inventors disclose technology development of a High Sensitivity DNA-linked Immunosorbent Signal Amplification Assay (DLISA) based on a high-affinity spike protein mini-binder, a nanobody, and controllable multiplexed DNA-fluorophore signal amplification using PER (FIG. 10). Our objective is to develop a SARS-CoV-2 antigen and virus detection assay capable of providing a quantitative, fast, low-cost approach to detect diverse variants of spike and viruses. Our preliminary results indicate that the nanobody (Nb3) and mini-binder have a high affinity for the spike protein and variants of concern, and that PER cascades can achieve highly multiplexed signal amplification probe via annealing with DNA-fluorophore. Inventors hypothesize that the development of a DLISA approach will be necessary for promoting VOC detection and increasing the sensitivity of antigen diagnosis test. Impact: This work will create a powerful new technique for VOC antigen detection and quantifying the concentration of whole virus particles. Optimizing biomolecules, such as the multiplex single strand DNA, DNA-fluorophore, mini-binder-DNA and nanobody, will ultimately result in a high-sensitivity, low-cost approach that avoids antibodies altogether.

To develop a point-of-care DLISA for rapid quantification of whole virus particles in clinical samples, inventors integrate DLISA with an imaging-based digital immunoassay to create a simple, yet sensitive and precise, point-of-care compatible device to measure SARS-CoV-2 levels in the sample. Given the strong fluorescent signal provided by the multiplexed DNA-fluorophore construct, inventors explore fluorescence-based digital counting of viral particles on the sensing surface.

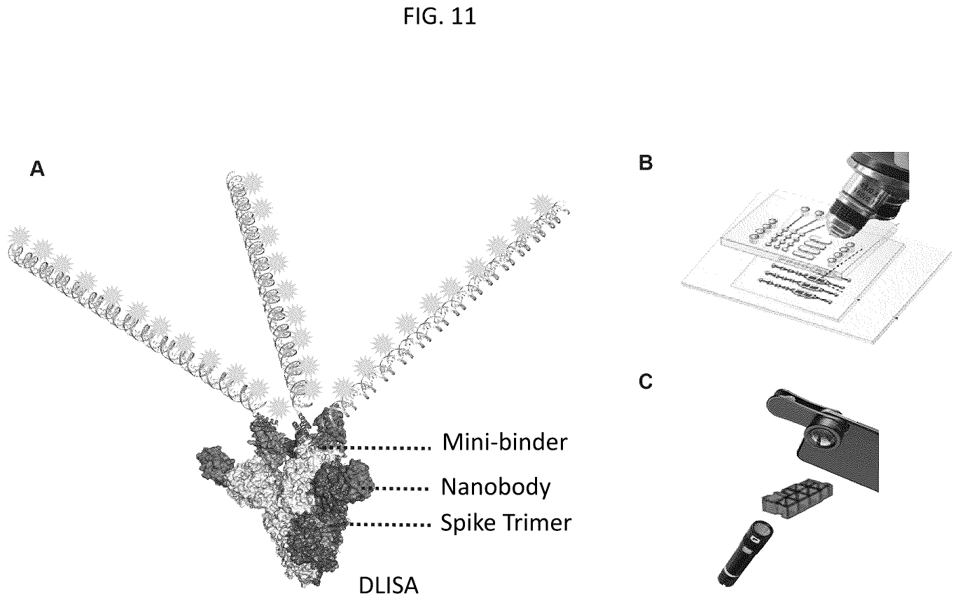

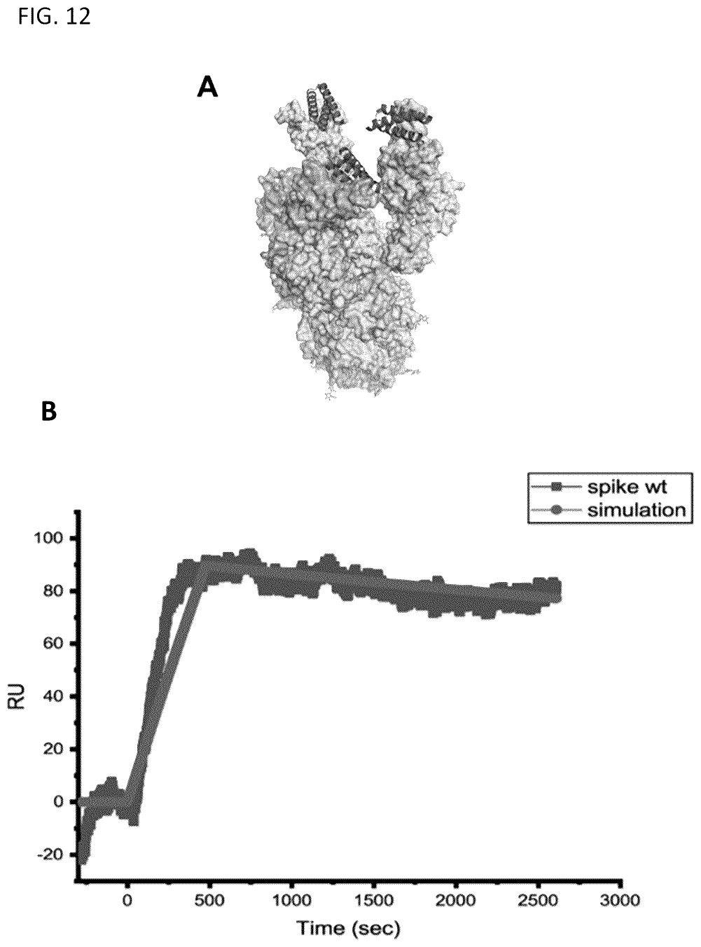

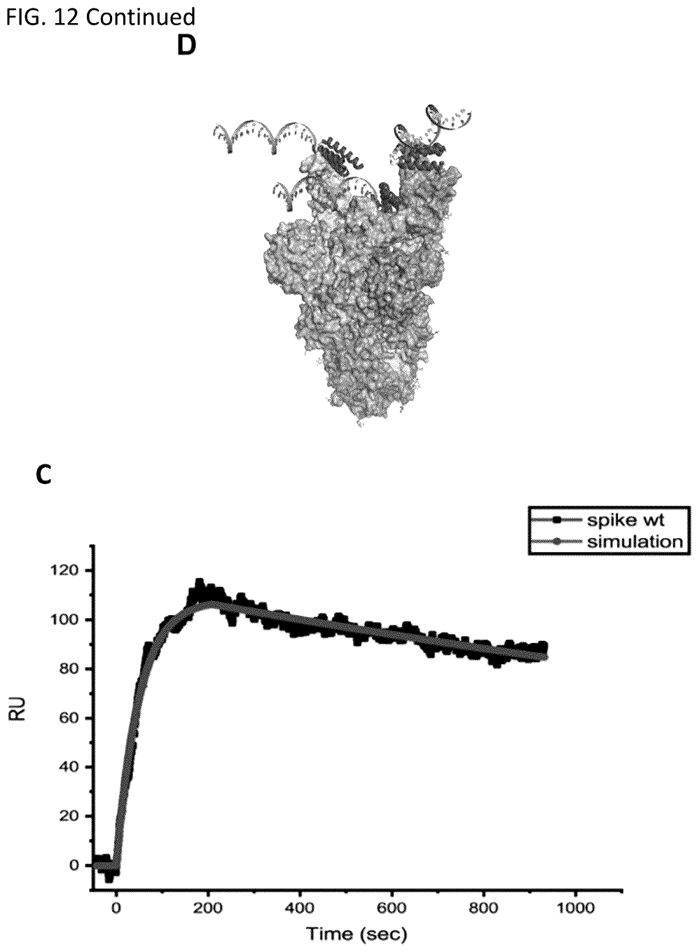

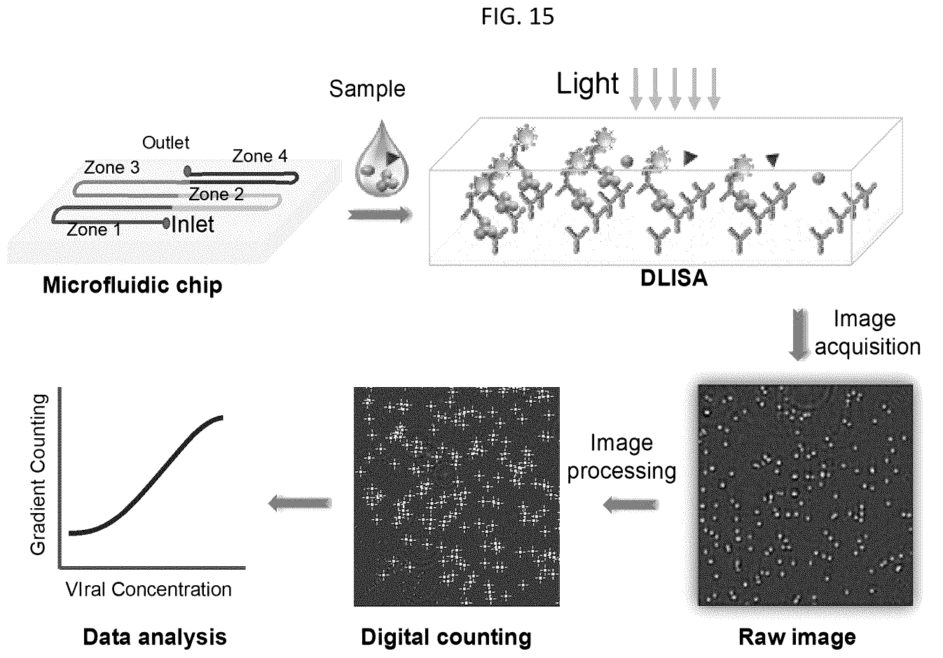

Principle of imaging-based digital DLISA: As shown in FIG. 11A-11B, the proposed digital immunoassay is carried out in a microfluidic chip and imaged with a homemade microscope for digital counting. The workflow of the digital immunoassay is shown in FIG. 15. By measuring different concentrations of spiked SARS-CoV-2 samples, counting the number of binding events to each zone, and using a gradient-based calibration method to subtract the non-specific binding effects, an analyte standard curve can be generated to determine the concentration of analyte in the patient's samples (plasma, saliva or other body fluid).

In the microfluid channel, the sample only occupies a single zone at a time. The analyte is pushed from one zone to the next by air injected into the inlet, with precise control of the time of the analyte in each zone. Due to the small sample volume and excess capture nanobody, binding to the sensor surface reduces viral particle concentration in the sample as it moves sequentially between zones. This leads to a decrease of viral particle density on the sensor surface along the flow direction, which can be described as a gradient or difference in the fluid direction between the zones. The level of viral particles in the sample is determined more accurately from the differential signals between two zones, rather than detecting only one single location or averaging the signals of the entire sensor surface. A more quantitative description of the principle can be found in Jing W, 2020 et al.

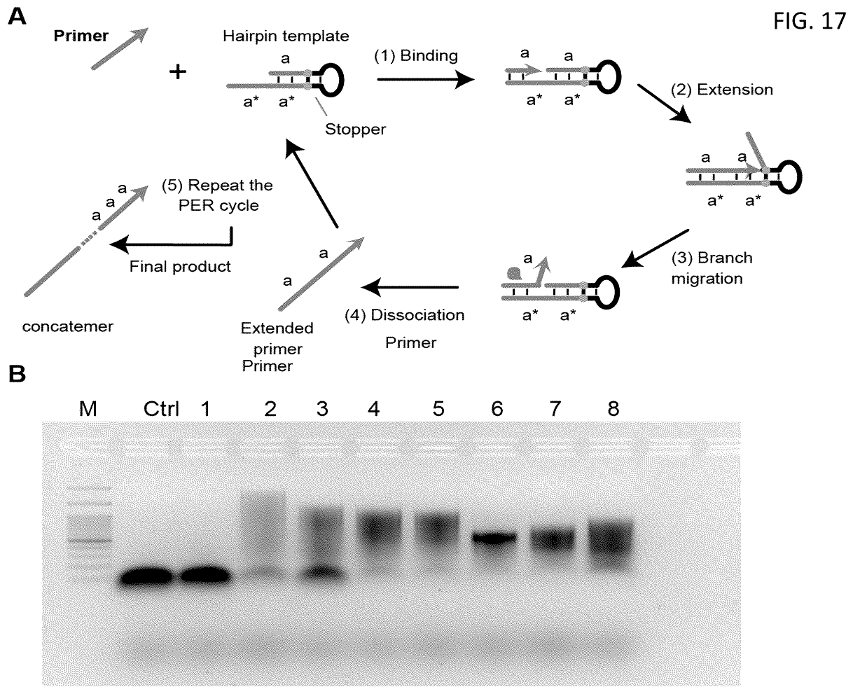

One aspect of this technology is to develop a highly sensitive & specific antigen detection assay, especially targeting the variants of spike protein, using a de novo designed mini binder with high-affinity of SARS-Cov-2 spike variants as a binding domain, and a concatemer single-strand DNA by PER cascades as a signal amplification probe. Although many commercial Ag-RDT tests for SARS-CoV-2 have been widely used for clinical detection, most of these rely on antibodies with high affinities for the target. However, emerging SARS-CoV-2 variants result in changes that render antigen diagnostic tests less sensitive or make them fail outright. In our preliminary data, inventors found that the mini binder can robustly bind with SARS-CoV-2 variants (FIG. 15), indicating that the protein can replace antibodies to improve the sensitive diagnostic detection of these variants. Immuno-SABER is a highly multiplexed and individually controllable signal amplification method by PER. As shown in FIG. 17, inventors have designed a short hairpin template and primer, successfully producing a 500-600 bp DNA length by the PER method. Our approach will allow us to detect any spike variant and SARS-CoV-2 virus, with signal amplification via controllable fluorescence, gold nanoparticles, etc. —all advantages not currently possible with existing Ag-RDT assay technology. Using self-assembling DNA will dramatically control the labeling with different signals such as fluorescent dyes. Our approach also significantly decreases cost (compared to antibodies) because both the mini-binder and the nanobody are obtained by recombinant expression in E. coli.

The imaging-based digital DLISA for detection of whole SARS-Cov-2 viral particle has the following innovations and advantages: 1) Detection of whole viral particles directly with simplified sample preparation; 2) Direct optical digital counting improves the detection limit and precision, while reducing the assay time. 3) Gradient-based differential detection significantly reduces signals from non-specific binding. Digital counting of individual viral particles provides ultimate sensitivity: If a 100 μL sample is used, the theoretical detection limit is 100 viral particle/mL. (Assume 100% capture rate, and detect 10 particles on the surface as threshold for positive infection), which is more sensitive than commercial rapid assays (10\hat{ }5 to 10\hat{ }8 RNA copy/ml for most rapid antigen assay).

Project Overview. The goal of this project (FIG. 10) is to develop a high sensitivity DLISA assay. As shown in FIG. 10, the sandwich DLISA assay includes nanobody coating on a plate as a capture domain, mini-binder conjugated with DNA-fluorophore as a signal amplification probe. The nanobody can capture the spike protein. The high-affinity spike RBD mini-binder with a signal amplification DNA probe will significantly enhance the sensitivity and specificity in the identification of SARS-CoV-2 variants of concern (VOC), which is an antibody-free assay.

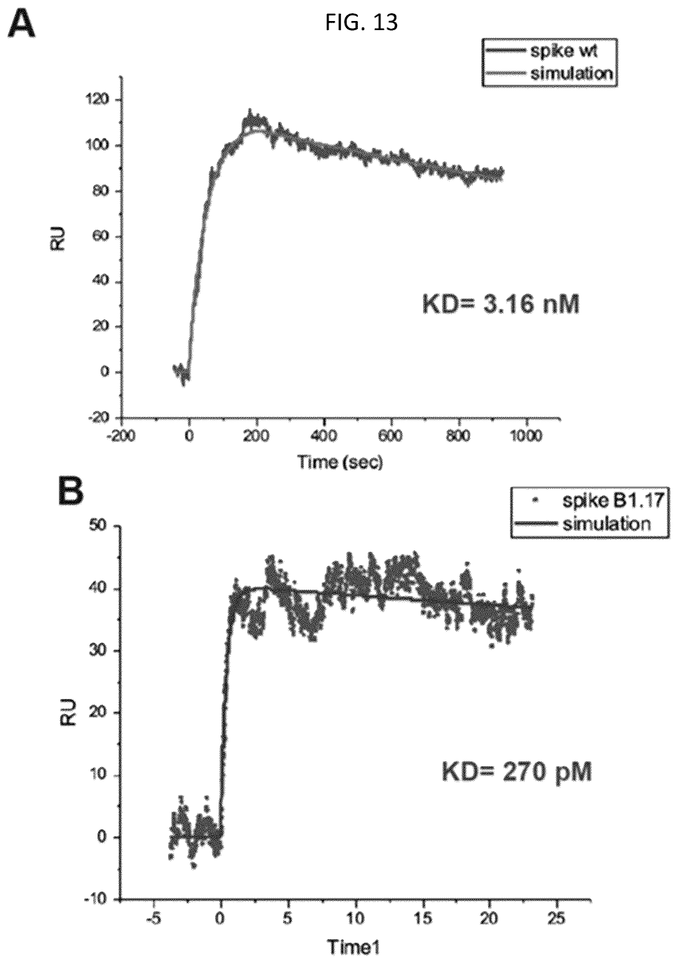

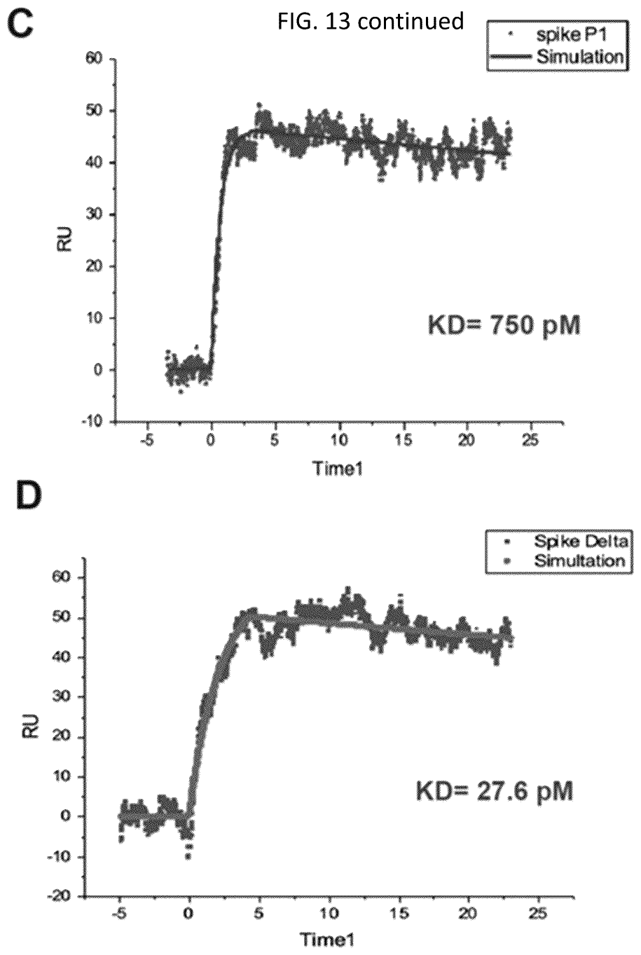

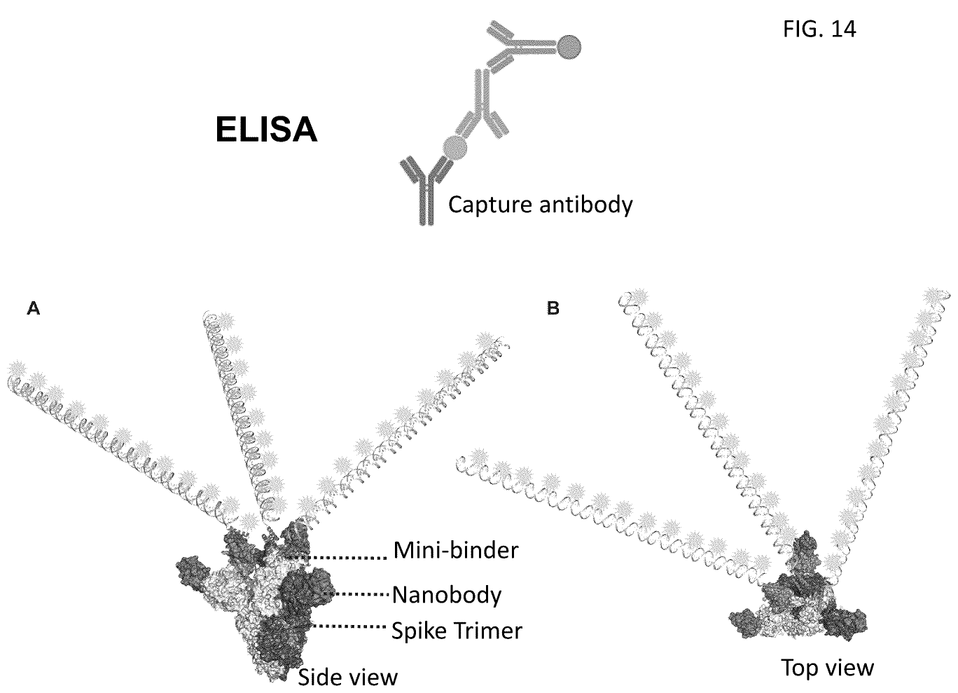

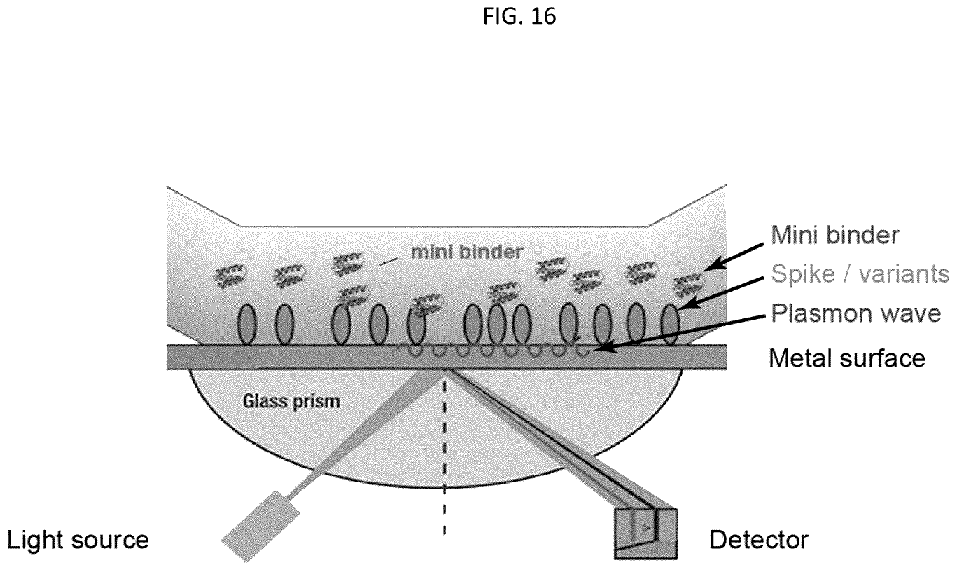

The mini-binder protein has a high affinity for spike variants. Surface plasmon resonance (SPR) is a powerful technique for studying the kinetics of mini binder & spike protein interactions (FIG. 16). To prove the interaction of the spike protein with the mini binder, the interaction kinetics were studied by a commercial SPR system (Biosensing Instrument SPRm200). The spike protein and variants such as alpha (B1.1.7), gamma (P1), and delta (B1.617.2). were immobilized on separate gold chips and mounted to the instrument. After flowing buffer to establish the baseline, 0.1 mM mini binder protein solution was introduced to the sensor surface to measure analyte association. As shown in FIG. 13B, 13C, 13D, 13E, the SPR signal increased exponentially and reached a steady state. Next, the analyte solution was replaced with PBS buffer to measure analyte dissociation. The slow decrease of the signal indicates a tight binding (low koff). The binding affinity is quantified by calculating the dissociation constant (K_{D}) via fitting the response curves with first order kinetics. Interestingly, the K_{D} value between the mini binder and the spike wild-type protein was measured as 3.2 nM, with the alpha spike as 270 pM, the gamma spike as 750 pM, and the delta spike as 27.6 pM. Our data indicated that the mini binder protein has high binding affinity to the diverse spike variants.

Primer exchange reaction cascades for synthesizing multiplex single DNA strands. For an antigen detection assay, DLISA relies on controlled in vitro synthesis of an amplified concatemer by PER. A PER reaction is patterned by a single catalytic hairpin template (FIG. 17A), which dictates the sequence (domain a) that becomes appended to primer sequence. FIG. 17A illustrates the PER cycle. In our experiments, a 9-nucleotide primer is used as the binding domain, which enables effective priming and permits efficient spontaneous dissociation. In step 1, a primer binds to its complement a* on the 3′ end of the primer. A polymerase will subsequently extend the primer in step 2 before halting at the stop sequence (black color). The copied a domain is able to compete with the domain on the hairpin template through the random walk process of branch migration (step 3). Finally, the extended primer will spontaneously dissociate from the hairpin template (step 4). Primers can be labelled with a dye on their 5′ terminal for tracking in gel electrophoresis (FIG. 17B). Our data showed that the ordered elongation of the primers was validated in gel electrophoresis in different reaction conditions. The synthesized 500 nt single-stranded DNA (ssDNA) concatemers (lane 6) was shown in FIG. 13B. The single strand DNA concatemer will anneal with DNA-fluorophore complementary strands as a signal amplification probe.

Preliminary results of imaging-based digital Immunoassay: Table 1 shows demonstrated imaging-based digital immunoassays for rapid and sensitive detection of blood biomarkers with high clinical values. Compared to existing rapid detection assay, this method includes very small sample volume, is point-of-care compatible, and outperform most commercial rapid detection assay on the corresponding protein biomarkers. Furthermore, the method is compatible with DLISA, and can be integrated to create a rapid SARS-CoV-2 antigen test.

TABLE 1
Performance of imaging-based digital immunoassays

| | | | | | Gradient-based | |
|---|---|---|---|---|---|---|
| | Time resolved digital | | One step digital | | digital | |
| | immunoassay | | immunoassay | | immunoassay | |
| Detection Platform | Surface plasmon resonance | | Bright field imaging | | Bright field imaging | |
| | imaging | | | | | |
| Sample type | Spiked human sera | | Spiked human plasma | | Patient plasma | |
| | | | | | sample | |
| Sample volume | 100 | μL | 10 | μL | 1 | μL |
| Analyte | Procalcitonin (PCT) | | Troponin I | | Troponin T | |
| Limit of detection | 2.8 | ng/L | 5.7 | ng/L | 1.8 | ng/L |
| (LoD) | | | | | | |
| Limit of quantification | 4.2 | ng/L | 6.6 | ng/L | 6.2 | ng/L |
| (LoQ) | | | | | | |
| Analytical time | 30 | min | 10 | min | 30 | min |

### Experimental Approach

### Development of DNA-linked Immunosorbent Signal Amplification Assay (DLISA)

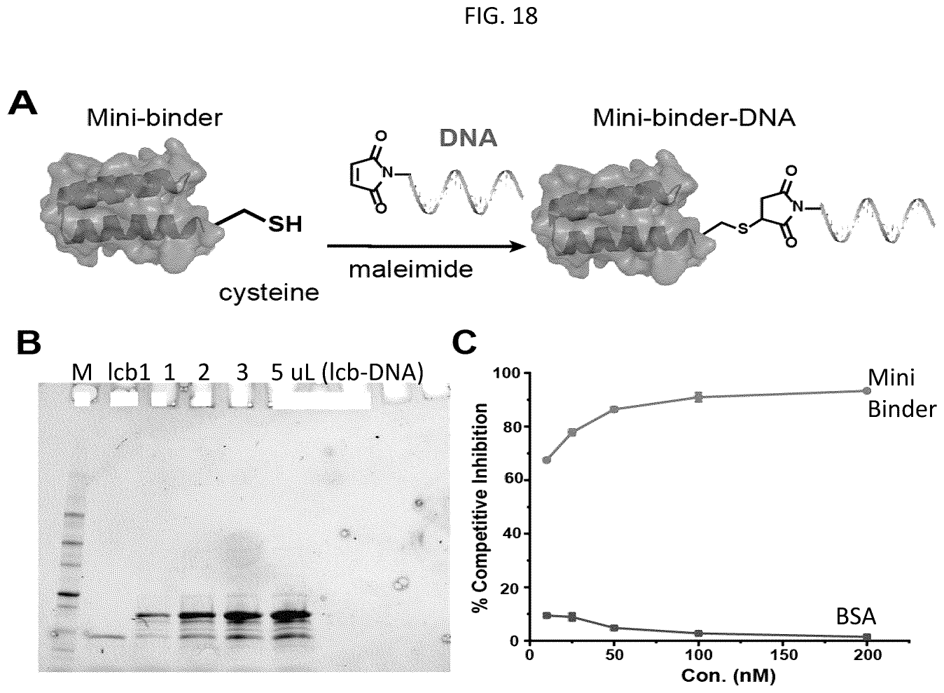

Synthesize a concatemer single strand DNA and mini binder-DNA probe. As indicated in FIG. 17A, a 500-nt single-stranded DNA (ssDNA) concatemers was synthesized, which are generated in a preprogrammed manner via primer exchange reactions, and which bind to complementary AlexaFluor488-DNA stands for signal amplification (FIG. 17B). (1) Inventors first verify the stability and optimize the fluorescence intensity of the DNA probe in vitro. The melting curves for probes will be measured on an Aviv 14DS UV-vis Spectrophotometer fitted with a thermoelectric temperature-controlled cell holder. The fluorescence intensity of the probes will be tested to evaluate the influence of fluorescence influence after Alexa488-bearing DNA stands hybridize to ssDNA concatemers to form the signal amplification DNA probe. (2) Inventors conjugate the mini binder with DNA. Inventors expressed and purified the mini-binder protein, reported by Cao et al, that targets the spike RBD with IC₅₀ values ranging from femtomolar to nanomolar. Preliminary data show that the mini-binder has a robust binding affinity up to the picomolar regime for spike variants (FIG. 13). The mini binder will be conjugated to DNA via a unique, mutagenically-introduced cysteine amino acid using a bifunctional linker maleimide (FIG. 18A). The cysteine-containing mini binder will be expressed recombinantly in E. coli. Conjugates will be purified using anion exchange chromatography and characterized via denaturing PAGE gel electrophoresis (FIG. 18B). Next, inventors assess the binding affinity constants and specificity of the mini binder-DNA conjugate using ELISA (FIG. 18C) and SPR assays. (3) The mini binder-DNA conjugate will hybridize to the DNA probe with multiple Alexa 488 fluorophore dyes (FIG. 10). The binding affinity, specificity, and fluorescence intensity of the mini binder-DNA probe will be assess using SPR, ELISA, and Synergy Neo2 plate reader.

Validation and characterization of DNA-linked Immunosorbent Signal Amplification Assay The sandwich DLISA assay consists of the captured nanobody, which targets N-terminal domain (NTD) of the spike protein with nanomolar affinity, and the high-affinity spike RBD mini-binder with a signal amplification DNA probe. (1) Optimization for nanobody concentration: In the first screen, inventors aim to define the concentration of nanobody coating on plate wells as a capture domain. The different concentrations and specificity of the nanobody will be assessed by ELISA to determine an optimum concentration for coating. (2) Development of the sandwich DLISA. To generate DLISA, the nanobody and mini binder DNA probe create a sandwich complex with the spike protein. The nanobodies will be coated on a 96 well plate as a solid phase. The commercial spike and variant proteins will be diluted to the different concentrations to add into well individually. After incubating the mini binder-DNA probe, the fluorescence intensity will be determine using the Synergy Neo2 plate reader. (3) Intrinsic sensitivity of DLISA. To assess the intrinsic sensitivity of DLISA, inventors assess the lowest analyte concentration (LOD) measured by DLISA, which can be reliably distinguished from the limit of blank (LOB). Inventors determine the LOB which is the highest apparent analyte concentration expected to be found when replicates of a sample containing no spike are tested. (4) Specificity of DLISA. To evaluate the cross-reactivity of DLISA, inventors measure the detection value for bovine serum albumin (BSA) and the SARS-CoV-2 nucleocapsid protein for both nanobody and mini binder against spike protein for both DLISA and ELISA.

Expected Outcomes and Alternative Approaches: Inventors' approach will yield high-affinity (sub-pM) and sensitivity mini binder DNA probe for the target protein, with neutralizing effects for Spike and variants. These probes will be optimized in terms of different fluorophores and gold nanoparticles, and can be applied to a microfluidic digital DLISA assay in a follow-up application. The risk in our approach is the stability of the probes. To address the risk, modified DNA such as phosphorothioates backbone could be used to improve the chemical stability.

### Development of Micro-Fluidic Digital DLISA Assay for the Detection of the Whole Virus Particles.

Produce pseudo-virus for imaging-based digital DLISA Assay. DNA has emerged as an exceptional molecular building block for nanoconstruction. Inventors develop pseudovirus to test performance using the imaging-based digital DLISA assay. The pseudovirus bearing the spike protein gene will be produced in 293T cells. The pCMVΔR8.2 and pCMV-spike plasmids will be co-transfected in 293T cells. Pseudovirus supernatants will be collected approximately 48 hours post-transfection and filtered through a 0.45 mm low protein binding filter, and immediately stored at −80° C. for the next assay step.

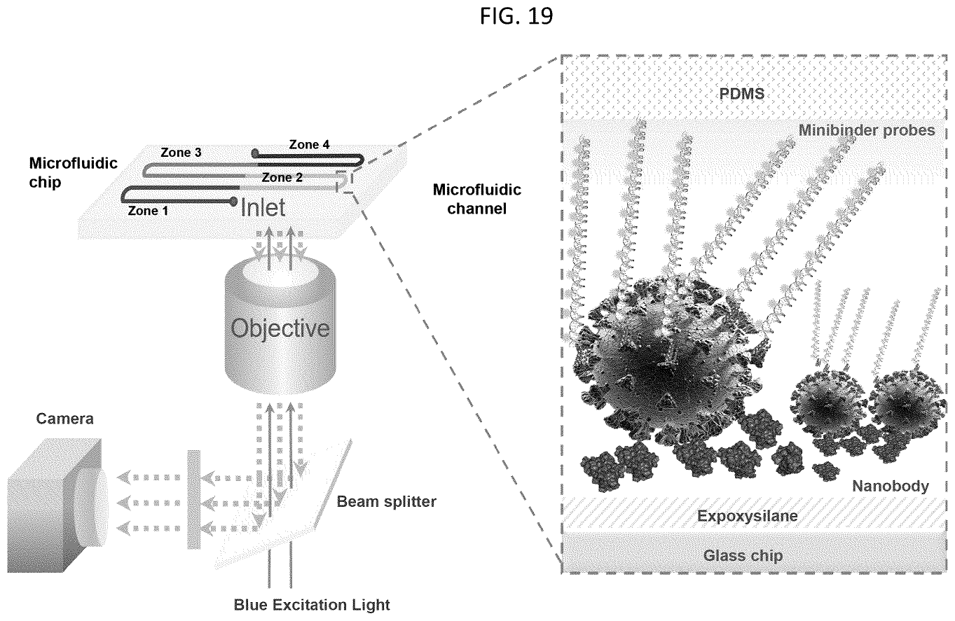

Develop imaging-based digital DLISA Assay. Based on the DLISA concept, inventors develop an imaging-based digital assay for point-of-care COVID antigen test. Inventors build and test a proof-of-concept prototype microfluidic device as illustrated in FIG. 19 to test fluorescence imaging-based digital counting of whole viral particles. To minimize the exposure risk and save time and cost, pseudovirus spiked in buffer, pooled human saliva and serum as model system could be used to establish the assay performance and sample preparation protocol. Inventors check if the fluorescence signal is sufficient to resolve individual viral particles bound to the sensor surface in the microfluidic channel. Assay conditions including nanobody surface density, minibinder probe concentration, illumination light intensity and camera exposure times will be optimized for accurate viral counting. Inventors will test one step assay first, means Minibinder probes is mixed with the viral sample and delivered to the microfluid chip in a single injection. This will provide the fastest assay time. If signal intensity or non-specific binding become an issue, two step assay (sample incubated with the chip first, then adding Minibinder probes) or gradient based assay could be used instead. After the assay format determined, a dose-response curve with different concentration of the viral particles in triplicate can be measured to obtain the specificity, sensitivity and resolution efficiency of the digital DLISA assay.

Evaluate and Validation of the digital DLISA assay with clinical samples. After establishing the basic assay performance with pseudo-virus, Inventors will validate the assay performance with patient samples. Inventors will obtain stored residual patient saliva, serum and nasal swap samples from the Biodesign COVID test center (see support letter) and assess the testing specificity, sensitivity and resolution efficiency in these SARS-CoV-2 samples, including both positive and negative samples (5 for each time of samples).

Expected Outcomes Alternative Approaches: Inventors expect to obtain proof-of-concept data on imaging-based digital DLISA assay for rapid point-of-care SARS-Cov-2 antigen test that works for all VOCs. In case the fluorescence-based single molecule counting encounters sensitivity issues, inventors may switch to gold nanoparticle labels for transmitted or scattering based imaging quickly given the flexibility of DNA construct. In this way, the signal to noise ratio are limited by shot noise, and can be improved with higher power illumination light and multiple frame image averaging.

Based on the results of the proposed research additional directions include DLISA and DNA probe modification for high sensitivity variants antigen test, and smartphone based portable imaging system development for point-of-care testing. Validation of the technology with additional clinical samples may occur. One of the major advantages of the proposed platform, is its utility for different types of the SARS-Cov-2 variants test and applications. As a result, the proposed research will also be expanded to include additional biomolecules and tissue staining applications, such as: SARS-Cov-2 infectious tissue and the study the virus distribution in tissue.

All of the referenced cited herein are incorporated herein by reference, for any purpose, in their entireties.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

We claim:

1. A method of detecting the presence of SARS-CoV-2 in a sample from a subject, the method comprising:
i) contacting the sample to a capture molecule, the capture molecule comprising a nanobody specific for SARS-CoV-2, wherein the capture molecule is linked to a solid support;
ii) incubating the sample in the presence of the capture molecule under conditions for SARS-CoV-2 in the sample to bind to the capture molecule, thereby forming a “V-AB” complex;
iii) contacting the V-AB complex with a detection molecule under conditions to allow the detection molecule to bind the V-AB complex, the detection molecule comprising a DNA-peptide hybrid molecule, the DNA-peptide hybrid molecule comprising a DNA nanostructure chemically linked to one or more target-specific binding peptides, wherein the target-specific binding peptides specifically binds SARS-CoV-2;
iv) detecting the presence of SARS-CoV-2 in the sample based on the presence of the bound detection molecule.

2. The method of claim 1, wherein the DNA nanostructure of the detection molecule comprises one of: a single-stranded DNA molecule, a three-helix bundle, a four-helix bundle, a six-helix bundle, a triangular DNA origami structure, a tetrahedral wireframe cage, a block-like origami cuboid, reconfigurable tweezers, double crossover tiles, branched three-way junctions, and a three-legged stool.

3. The method of claim 1, wherein the DNA nanostructure is linked to more than one target-specific binding peptide.

4. The method of claim 1, wherein the DNA nanostructure is linked to three target-specific binding peptides.

5. The method of claim 1, wherein one or more of the target-specific binding peptides comprises LCB1.

6. The method of claim 1, wherein the capture molecule comprises a nanobody that specifically binds to the N-terminal domain of the SARS-CoV-2 spike protein.

7.-12. (canceled)

13. The method of claim 1, wherein the DNA nanostructure comprises a single stranded DNA molecule.

14. (canceled)

15. The method of claim 13, wherein detection comprises a primer exchange reaction (PER).

16. The method of claim 15, further comprising contacting the detection molecule with fluorescently labeled oligonucleotides that hybridize with the product of the PER.

17. The method of claim 1, wherein the target-specific binding peptide binds SARS-CoV-2 alpha, beta, gamma, and delta spike protein variants.

18. The method of claim 1, wherein the method further comprises treating the subject based on the detection of SARS-CoV-2 in the sample.

19. A kit for detecting the presence of SARS-CoV-2 in a sample comprising:
i) a capture molecule linked to a solid support, wherein the capture molecule is a nanobody specific for SARS-CoV-2;
ii) a detection molecule comprising a DNA nanostructure linked to one or more target-specific binding peptides, wherein the one or more target-specific binding peptides bind SARS-CoV-2.

20.-31. (canceled)

32. A method of detecting the presence of SARS-CoV-2 in a sample from a subject, the method comprising:
i) contacting the sample to a capture molecule, the capture molecule comprising a DNA-peptide hybrid molecule, the DNA-peptide hybrid molecule comprising a DNA nanostructure chemically linked to one or more target-specific binding peptides, wherein the capture molecule is linked to a solid support;
ii) incubating the sample in the presence of the capture molecule under conditions for SARS-CoV-2 in the sample to bind to the capture molecule, thereby forming a “V-AB” complex;
iii) contacting the V-AB complex with a detection molecule under conditions to allow the detection molecule to bind the C-AB complex, the detection molecule comprising a SARS-CoV-2 specific binding molecule (SBM);
iv) detecting the presence of SARS-CoV-2 in the sample based on the presence of the bound detection molecule.

33. The method of claim 32, wherein the detection molecule comprises a nanobody specific for SARS-CoV-2.

34. The method of claim 32, wherein the DNA nanostructure is selected from the group consisting of: a single-stranded DNA molecule, a three-helix bundle, a four-helix bundle, a six-helix bundle, a triangular DNA origami structure, a tetrahedral wireframe cage, a block-like origami cuboid, reconfigurable tweezers, double crossover tiles, branched three-way junctions, and a three-legged stool.

35. The method of claim 32, wherein the DNA nanostructure is linked to more than one target-specific binding peptide.

36. The method of claim 32, wherein the DNA nanostructure is linked to three target-specific binding peptides.

37. The method of claim 32, wherein one or more of the target specific binding peptides are LCB1.

38. The method of claim 32, wherein the capture molecule binds to the receptor binding domain of the SARS-CoV-2 spike protein.

39.-46. (canceled)

47. A kit for detecting the presence of SARS-CoV-2 in a sample comprising:
i) a capture molecule comprising a DNA nanostructure linked to one or more target-specific binding peptides, wherein the one or more target-specific binding peptides bind SARS-CoV-2, wherein the capture molecule is linked to a solid support;
ii) a detection molecule comprising a SARS-CoV-2 specific antibody (SAB).

48.-58. (canceled)


SEQUENCE LISTING (5 sequences)

SEQ ID NO: 1 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 56 aa, synthetic construct
DKEWILQKIYEIMRLLDELGHAEASMRVSDLIYEFMKKGDERLLEEAERLLEEVER

SEQ ID NO: 3 - protein, 6 aa, synthetic construct
  VARIANT 5..6
GPRPXX

SEQ ID NO: 4 - DNA, 14 bp, synthetic construct
  source 1..14: mol_type = other DNA
ctgaatggtacgga

SEQ ID NO: 5 - protein, 23 aa, synthetic construct
IEEQAKTFLDKFNHEADLFYQSC